SEQUENCE LISTING (81 sequences)

SEQ ID NO: 1 - DNA, 5233 bp, Homo sapiens
cttgctggccctggggaagtatcttgactttttttctataagaattgggaagctccaaaagctctgaatagtgataggagcagaacattgtaccagaaagattagtgtaattgtactgataattgattgagggagtcaaccaattgataggtggatgatattgtacaagcctagacaaaaggtgatgagggcacccattagttcatcgccacttggtcccttcatcattagtacttctctgccagagacatctgtttatttgtattgtaattatttaacttgtctctctccttttcttcactaataatgtagcacatttagcactggagctagacacttctaattatcccccaatattccttggctacagtaataaaacattgtgagtttgagccggacacagagctaccagttaaagactacatgtcccagcctctcttgcaactagctgtggccataagactaggttttggcaatggatttgagcaggagtgaggattgctgtttctgggacatgccctcatagtgaagctgtttgctcttcatttcttcctgcctcattcttgcagattgctccatacccatttttctctcctccacctgaagtaggtgttggagatgatgcccttttggaactacatagcttccttcatccttttcctgagagacaggtacgtgggcttgggagttgcttcatgggtcaagctttcataggcttttgcaaaaagggaaaatgtaggtgtatttattactaggttctggccagttggatgagaatgaaagtggggtgctgtgtctgggtcatgcacataatgggaagctctctgcttccactttcctccttcttgagggcaggtatttgaccctggtggtctcgagccccccttgtctcatgtgtcattttacaggatgcaaagaacaaacatgctcctatgcccctgcacctgcctcatggggaatgctgccaacctcctgggattgcctacaaaattgaacttctattatgagagaaacaacttccatcttaattaagctattgttatttggtcagcgttacagatgccaaattaatattttaatatataatttataaatattgatgattaccactacgtgttaaatgagcacatgattggtaaatataaaacacactatacatgtaaagtataggttgaactatcccttatctgaaatgcttgggaccagaagtgttttggatttgtttttgttttttgttcaaatttggaatacagtcatccctcagtctccatgaggaattgtttccaggacctcctgcagataccaaaatcttcagatgctcaagtccctgatataacatggcgtagtatttgtatgtaacctacacatttctacctatatactttaaatcatgtctatattacttataatatctaaaacaatataaatgctccataaatagttgctatgctctattgtttagggaataatgactcaaaaaaaaaagtctgtacatgttcaatacagataaaatttttatcccaaacatttcaatccaaggttagttgaatccatggaggcagaatctgtacaaagagctgactatatctgtattatatactgatggtcacgccaggttctgaaaatctgaaatccaaaatgctcaaaagagttttctttgagtgtaatgtcaacactcaaaaagttttgtattttggaccacttcaggtttcagacttttggaataggaatactcaacctttaatattaaaaaaattatttattccattcccctaaactttgagcataaagcatctatagtcttttgagaaggaaagctgtaatagaaagcttaggctgaactcaacttctctgtcaccataaccctgggtcgattttctaacttgcttcgtgataagcttgttcagcacagaaagtatctgacaagataaagacaacaaaatactgggttactagtttctggtcatgggctctatcacaccccaaacaagacttttaaaggaaaatgaactatgaaatcttcaagttgtgtatctcatatctctttataggcaagcactataaaaatgtgaatttgaatattatttcattgcagggcttttgtgatgcttgttttcttatagaatgcaacaaaaatttcatggaaagaaagtctagtttctattgaagaaaaatatttgacattgagattttaaaaattttgccatttacatttatcattattttttcaataatcttgcactctcatatccagattgtttaaataagcatttctgcttgcacagcccatttgatgaaacatatttattatcaagtttatgtacttgtatcactatcgcactcagagaaatatcaggagtctctgtataactccttatgatatatacagttcttcatgttttaggtggaagagcattggaatgttgacttatctaactggaaaagtggtttggagggtgttggctcttggagcatgaggttgcaattaagaaaagctggaaattggcatacacgtcctgaatcaaaatacaccttcagaaagagatgaggacattttcaccttataatgctgagaagtctatactgctaaagataaaaatggtgaaatgtaaatattggttatgaaattgaaaatttttatttcctgtaccactgaagttattttgtataaacggtattgtccagccttctttttatcaagatttgaatgttgttattttggttttcctcggagtactaagggcagggactttgctttgctgatcccaaatcccagaactgtgcttagcaaacactgggtattaaaaaaaaaaaagagagagccagttgttgactgaataaatagatgaatggataaataatgtttgcatttaagaattacgatttccaatggcaagagaggtattgctagtacaagattttcctttagaacataaaaagagaagataatggatctcaattaagttgtttataaagaagcctgcttcataatcaatgttttttttaagtcatgtaggcatacttattacatttggcaatacagaaacatcagattttgcagaactatctctttaggtgtaagattatattaaagaattaatatgatacaagaattatgaatacaggtttaggaaaaaacagaaaagaaccccaaccagtaaaaaaaaaattaaagtataacattaaaaaacatcaaaattgtaaatattgtgtagaagaaaaactaaatgattaacctgaatggttatggtattgctgataaatgcatcatcttgactcctaggagaaccaatttatgtgaaattccatgaaaaagaattagttacaacaagcagaattttagtccatttccaagaattttaactactgtaaatcccctgacacacctcccaaataattaggatatcgttttgcaatagccacatgggaacctggccctagaggtctataggtaatctgtttcattcatgtattttaagtatgtcgtttaggaataagttatcaggtttgcaacctataagcaaaggaaataatgtgacactggaaaacaacactattcatttaacataatgaattgccatgtaataactcagatcttcccagggtgtaaattacacaaatttgaaagatgcatttattatttaatgcctcattcccagacagttgcttactcagtagcaaaatctgtcttagcataccaagtgtaaagctatttaacaaataggaaggtttaaaaaatatatactatcatgcagacagctaaaatatttgtatatatttttaatcttttttctctaatgatacttagaatattttatttttattactacaaataatagagatgaaatatgaattgtattagtagcagagatatatgagctaaagcttgtattgtttaaagcacatcatcttaaaaggcctgtcaggaaacagtgttcatattaagttggctttcagtactctaagaagatgacatcattttgtaagagacaagtgttgttagagcaaatgctaggatattctaaaatttcctaggttgaagtgaagaaatttctcattatagattatttcatgagtttatgttcccggttgtatatcagctcatgttaaattttgcaagagtttatgatttctaagaactcaccttctataaggccctttgctgagtggggctagttaggaattagtaagtaaaggggatcttttttcctcgtgtaaatagcttaagagtaattttgggcggtccagaaaccataagttatcaggaaggtgcttataaatgggcagagtacatcacttgcccaagattctaacaacctagcctgccccccacacactgtggggcaccgtttgctacttgccaagtaactgccttttttggcaaagaccacccaggacatggctcagagtccatcctaaggctggccaacctctgtaaatctcgtgtcccctgattcagagcgagtgcatttaattcaggaagatcacttacgactgagtttttcatcatggctttgtctgtgaaaccctcagaaaccagagagtgaggctggtgcaccgggagcggctgttgtgccagcagcttggtcctcttcggcatcttgtctgggcatttgtttaagctcagggtctctttttctgccaccatcttcctagaaaatgtcttgttctcataaaaagtgtagtaaaagaatcagtgggctttacggatgtgagcaggaggtctggaaaaaaatatcaaaaggcgcgataatggtagaaattcaacccctcgtagtgcccaggttgatccagatgtttggcagctcctaggtgaagggagctggaccctaggggcggggcgggaagagggcaggaccttggctagagctgcaacaagcttccaaaggtaagcctcccggttgctaagcgactggtcaacacggcgggcgcatagccccgccccccggtgacgtaagattgctgggcctgaagccggaaagggcggcggtggggggctgggggcaggaggcgtgaggagaaactacgcgttagaactacgactcccagcaggccctgggccgcgccctccgcgcgtgcgcattcctagggccgggcgcgggggcggggaggcctggaggatttaacccaggagagccgctggtgggaggcgcggctggcgccgctgcgcgcatgggcctgttcctggcccgcagccgccacctacccagtgaccatgataggtacgtgggta

SEQ ID NO: 2 - DNA, 400 bp, Homo sapiens
gtgcattgatatttaggcttcttgcccgaaggcactcctgtcttcaagaatttcctgtaccgacgtacagggaacataaactctgatcccagtaatagaaagctgagattaaacgcctttcctctttgtttccccaggcctacagagtcgatgaaagagccgccgagcaggctcgttgggaagcagcctccaaakaaaccatcaagaaaaccaccaagccctgtccccgctgccatgtaccagtggaaaaaaatggtgagtctgtgctgagcagagaatgaggatgtcgtgtgctctttgggggagaatcatacccatcagggattccaggattaaaggagatgctgtctgaaggtgcctggtgtgttgggtaacccctcgattaatgttacccattatc

SEQ ID NO: 3 - DNA, 1954 bp, Homo sapiens
cccctttcaggagaataaagtcagatttacaaataaaatttgttcccgacaaaagtgacatgcttcaatttcattcatttcttaatgaatatcatcactttagagctgccctattgtgctttatgaagtttttcccctcagttaagtttctctctgcccttgtattgcttgtgattattcgctcagaaagtgatgtctaggctagcgtgctggtttgggaatgcgtgttttccaggtacttgctgcgaacccaccacacctttgttttctgcccccaacaggaggctgcatgcacatgaagtgtccgcagccccagygcaggctcgagtggtgctggaactgtggctgcgagtggaaccgcgtctgcatgggggaccactggttcgacgtgtagccagggcggccgggcgccccatcgccacatcctgggggagcatacccagtgtctaccttcattttctaattctcttttcaaacacacacacacacgcgcgcgcgcgcacacacactcttcaagtttttttcaaagtccaactacagccaaattgcagaagaaactcctggatccctttcactatgtccatgaaaaacagcagagtaaaattacagaagaagctcctgaatccctttcagtttgtccacacaagacagcagagccatctgcgacaccaccaacaggcgttctcagcctccggatgacacaaataccagagcacagattcaagtgcaatccatgtatctgtatgggtcattctcacctgaattcgagacaggcagaatcagtagctggagagagagttctcacatttaatatcctgccttttaccttcagtaaacaccatgaagatgccattgacaaggtgtttctctgtaaaatgaactgcagtgggttctccaaactagattcatggctttaacagtaatgttcttatttaaattttcagaaagcatctattcccaaagaaccccaggcaatagtcaaaaacatttgtttatccttaagaattccatctatataaatcgcattaatgaaataccaactatgcgtaaatcaacttgtcacaaagtgagaaattatgaaagttaatttgaatgttgaatgtttgaattacagggaagaaatcaagttaatgtactttcattccctttcatgatttgcaactttagaaagaaattgtttttctgaaagtatcaccaaaaaatctatagtttgattctgagtattcattttgcaacttggagattttgctaatacatttggctccactgtaaatttaatagataaagtgcctataaaggaaacacgtttagaaatgatttcaaaatgatattcaatcttaacaaaagtgaacattattaaatcagaatctttaaagaggagcctttccagaactaccaaaatgaagacacgcccgactctctccatcagaagggtttatacccctttggcacaccctctctgtccaatctgcaagtcccagggagctctgcataccaggggttccccaggagagaccttctcttaggacagtaaactcactagaatattccttatgttgacatggattggatttcagttcaatcaaactttcagcttttttttcagccattcacaacacaatcaaaagattaacaacactgcatgcggcaaaccgcatgctcttacccacactacgcagaagagaaagtacaaccactatcttttgttctacctgtattgtctgacttctcaggaagatcgtgaacataactgagggcatgagtctcactagcacatggaggcccttttggatttagagactgtaaattattaaatcggcaacagggcttctctttttagatgtagcactgaaatccttgctggagggaagagaggggatgaactcaagttttccacatcctgggacacctgtccctcttttcctaactgcctaagataacccatttcttccaa

SEQ ID NO: 4 - DNA, 660 bp, Homo sapiens
ggaaaacgaacaggtttggagcaaaatgtcaaatatcgtctttgtatgttgatgaacatagttttgacctagcacatcccttgaaagggtcacggggacccccagagtctgcagaccacactttgaaaatcattggactacacactaatttcactattattttataacataagtggaaacatgtcttaaggagtacatttctattataactcatataagcatatattgttgttttttcccaaagggtccatcttgctgggatgatgttttaattccaaaccggatgagtggtgaatgccaatccccacactgccctgggactagtgcagwaagtacctggtcacmttcattcctcttattgcaagaaaatgatgacatcttcactgtttgccaggaaattagagacaaaatgtcaactgactgttcttccatctaataatgtttgccaaaagtgttatgatatttaaataggttaattacattcaccaaaattccaacctgtgcccctgcctttcagggtcactttcctagtgacttaatcatttggggggaccgtgtggaaatgtgccaatttaaactcattgcaaagttatatccatagaaggaaaaggagaggtgagaaaaggagagccagtgcagagcctccaaaagaaaaattac

SEQ ID NO: 5 - DNA, 500 bp, Homo sapiens
ttttgtggttggttagatgtgtgtttttcaggtacacgtctgtgtcctcccaaaaggcaacactggcagttgatagtcataactctgtgtaagaacatataaccacacagagtgaaagtgacgtttttgtgattaattcttctttccaacagctggctgtcccaactccttgattaaagagctccatcacttcaggattctgggagaagagcagstgagtgagcatctcaaaggctgcatcagactgtcatgaaagatagacgctaatgagacagtttgggctccccagggaggccgagtatgtctcctgaccctgggtgccctgaaatggggaagaaaaccatgctggagatatgtgtgaggacacttttttcctcttctatccatcagacctgacaggttattaattgctacatctgctatctgccagtgcagtgcatttcatctcaagactcaagcaggaagcaagcactgcatagtggcagatgagcaaacaaata

SEQ ID NO: 6 - DNA, 650 bp, Homo sapiens
taggagaatcagttttctatgtagttcattgagtgcctccaatttttaagatgttgtgttggtatacatgagcttaatgcttagcagctccggtctttgcacagagcacagtctacacaaccctccaggattacagaaattggtctaaagcacgtgctgcctttccacactgacaggtactagaggaaacatcttcctttctctctgcaggagccccgtcctggttttccagtgcaactcccgccacgtgatttgcttagactgtttccacttatactgtgtgacaagactcaatgatcggcagtttgttcacgaccctcaacttsgctactccctgccttgtgtgggtaagtctagcatgttttctctccatctctaatgctaatgaagaacagaagaacaattattgatgtaaaactggcttagatatacgtaaaccctagcagaagaatttaaatttgatcattgctggatatgaaacattaatgtttggatcgcaaaagataaaagttctggggaatgaaggaattgtgttgaactggaaaatgcattatttgcataaaggcattgagaataagtttgtcaatattattcagccaaggtatactaagtttttctgtgggttagagtcactctccatgttctagatt

SEQ ID NO: 7 - DNA, 700 bp, Homo sapiens
ggagaatgcaattttggtttgcaggtcactgacgaatatatgaaagggaaatctcgtgggtaactaactctgtttttcccaaatattgctctatagcattaagttttttgttgtaagtgaaagaaaatatataccattcactgaagggctgcgaggggtaaatcggttgagaaatgttgctatcaccatttaagggcttcgagtgatgctcactttctcttctcccttccaatttccttggtcagtgtttgtcaggttcaactccagccatggtttcccagtggaggtcgattctgacaccagcatcttccagctcaaggaggtggttgctaagcgacrgggggttccggctgaccagttgcgtgtgattttcgcagggaaggagctgaggaatgactggactgtgcaggtgagtctcccttggcggccgttcttgggatgccgccagctccattgctcatgccgcctgcgctgccaatctgacattcatgcctgagatctaatagaataaatagtgcctggggattccttgaactttactccacactgcttcattaattctgaccttcttaattatgcattaaaacagcaagcaggaaagattggaagaacaactgcgagtgagaaagagagagagaaagaacacacgagctaggcttagtgaataaatgtctactgactacaggagcagcaaggcaca

SEQ ID NO: 8 - DNA, 703 bp, Homo sapiens
tccttttgaatatgacgtcagcattctattgtgtttcacgtattcccaaatttctgtttctggccccagttcagtgttgtttgtctaccgtgtgtagtgtgtaactgctgtgggcaaaggagcacctaagttggtcagttacatgtcacttttgcttcccttctaccacggagggcaagttaaactctatctcgcatttcatgtttgacatttcctttttttttttttttttttttaccttgctcccaaacagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccaccagctggaagtccaggtaattggaatgctctaagattattaaagcattttgtttgtttgtttagtgcagtctgcatggagcatggcctcaccgggtgcatatttagtttatgatacgttttggattggagtctctaattcactacaaggagacatcactgtaggtggagtactttgatgtaacatttgagaatgcatttattgtaagtactatgaaacagg

SEQ ID NO: 9 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 10 - DNA, 610 bp, Homo sapiens
taagctcagggtctctttttctgccaccatcttcctagaaaatgtcttgttctcataaaaagtgtagtaaaagaatcagtgggctttacggatgtgagcaggaggtctggaaaaaaatatcaaaaggcgcgataatggtagaaattcaacccctcgtagtgcccaggttgatccagatgtttggcagctcctaggtgaagggagctggaccctaggggcggggcgggaagagggcaggaccttggctagagctgcaacaagcttccaaaggtaagcctcccggttgctaagcgactggtcaacacggcgggcgcatagccccgccccccggtgacgtaagattgctgggcctgaagccggaaagggcggcggtggggggctgggggcaggaggcgtgaggagaaactacgcgttagaactacgactcccagcaggccctgggccgcgccctccgcgcgtgcgcattcctagggccgggcgcgggggcggggaggcctggaggatttaacccaggagagccgctggtgggaggcgcggctggcgccgctgcgcgcatgggcctgttcctggcccgcagccgccacctacccagtgaccatgataggtacgt

SEQ ID NO: 11 - DNA, 2960 bp, Homo sapiens
tccgggaggattacccaggagaccgctggtgggaggcgcggctggcgccgctgcgcgcatgggcctgttcctggcccgcagccgccacctacccagtgaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacaccagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagccagggcggccgggcgccccatcgccacatcctgggggagcatacccagtgtctaccttcattttctaattctcttttcaaacacacacacacacgcgcgcgcgcgcacacacactcttcaagtttttttcaaagtccaactacagccaaattgcagaagaaactcctggatccctttcactatgtccatgaaaaacagcagagtaaaattacagaagaagctcctgaatccctttcagtttgtccacacaagacagcagagccatctgcgacaccaccaacaggcgttctcagcctccggatgacacaaataccagagcacagattcaagtgcaatccatgtatctgtatgggtcattctcacctgaattcgagacaggcagaatcagtagctggagagagagttctcacatttaatatcctgccttttaccttcagtaaacaccatgaagatgccattgacaaggtgtttctctgtaaaatgaactgcagtgggttctccaaactagattcatggctttaacagtaatgttcttatttaaattttcagaaagcatctattcccaaagaaccccaggcaatagtcaaaaacatttgtttatccttaagaattccatctatataaatcgcattaatcgaaataccaactatgtgtaaatcaacttgtcacaaagtgagaaattatgaaagttaatttgaatgttgaatgtttgaattacagggaagaaatcaagttaatgtactttcattccctttcatgatttgcaactttagaaagaaattgtttttctgaaagtatcaccaaaaaatctatagtttgattctgagtattcattttgcaacttggagattttgctaatacatttggctccactgtaaatttaatagataaagtgcctataaaggaaacacgtttagaaatgatttcaaaatgatattcaatcttaacaaaagtgaacattattaaatcagaatctttaaagaggagcctttccagaactaccaaaatgaagacacgcccgactctctccatcagaagggtttatacccctttggcacaccctctctgtccaatctgcaagtcccagggagctctgcataccaggggttccccaggagagaccttctcttaggacagtaaactcactagaatattccttatgttgacatggattggatttcagttcaatcaaactttcagcttttttttcagccattcacaacacaatcaaaagattaacaacactgcatgcggcaaaccgcatgctcttacccacactacgcagaagagaaagtacaaccactatcttttgttctacctgtattgtctgacttctcaggaagatcgtgaacataactgagggcatgagtctcactagcacatggaggcccttttggatttagagactgtaaattattaaatcggcaacagggcttctctttttagatgtagcactgaaa

SEQ ID NO: 12 - DNA, 2955 bp, Homo sapiens
ggatttaacccaggagaccgctggtgggaggcgcggctggcgccgctgcgcgcatgggcctgttcctggcccgcagccgccacctacccagtgaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacaccagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagccagggcggccgggcgccccatcgccacatcctgggggagcatacccagtgtctaccttcattttctaattctcttttcaaacacacacacacacgcgcgcgcgcgcacacacactcttcaagtttttttcaaagtccaactacagccaaattgcagaagaaactcctggatccctttcactatgtccatgaaaaacagcagagtaaaattacagaagaagctcctgaatccctttcagtttgtccacacaagacagcagagccatctgcgacaccaccaacaggcgttctcagcctccggatgacacaaataccagagcacagattcaagtgcaatccatgtatctgtatgggtcattctcacctgaattcgagacaggcagaatcagtagctggagagagagttctcacatttaatatcctgccttttaccttcagtaaacaccatgaagatgccattgacaaggtgtttctctgtaaaatgaactgcagtgggttctccaaactagattcatggctttaacagtaatgttcttatttaaattttcagaaagcatctattcccaaagaaccccaggcaatagtcaaaaacatttgtttatccttaagaattccatctatataaatcgcattaatcgaaataccaactatgtgtaaatcaacttgtcacaaagtgagaaattatgaaagttaatttgaatgttgaatgtttgaattacagggaagaaatcaagttaatgtactttcattccctttcatgatttgcaactttagaaagaaattgtttttctgaaagtatcaccaaaaaatctatagtttgattctgagtattcattttgcaacttggagattttgctaatacatttggctccactgtaaatttaatagataaagtgcctataaaggaaacacgtttagaaatgatttcaaaatgatattcaatcttaacaaaagtgaacattattaaatcagaatctttaaagaggagcctttccagaactaccaaaatgaagacacgcccgactctctccatcagaagggtttatacccctttggcacaccctctctgtccaatctgcaagtcccagggagctctgcataccaggggttccccaggagagaccttctcttaggacagtaaactcactagaatattccttatgttgacatggattggatttcagttcaatcaaactttcagcttttttttcagccattcacaacacaatcaaaagattaacaacactgcatgcggcaaaccgcatgctcttacccacactacgcagaagagaaagtacaaccactatcttttgttctacctgtattgtctgacttctcaggaagatcgtgaacataactgagggcatgagtctcactagcacatggaggcccttttggatttagagactgtaaattattaaatcggcaacagggcttctctttttagatgtagcactgaaa

SEQ ID NO: 13 - DNA, 10 bp, Homo sapiens
ggcctggagg

SEQ ID NO: 14 - DNA, 12 bp, Homo sapiens
tccgggaggatt

SEQ ID NO: 15 - DNA, 24 bp, Artificial Sequence
  primer
gcgcggctggcgccgctgcgcgca

SEQ ID NO: 16 - DNA, 20 bp, Artificial Sequence
  primer
gcggcgcagagaggctgtac

SEQ ID NO: 17 - DNA, 23 bp, Artificial Sequence
  primer
atgttgctatcaccatttaaggg

SEQ ID NO: 18 - DNA, 23 bp, Artificial Sequence
  primer
agattggcagcgcaggcggcatg

SEQ ID NO: 19 - DNA, 19 bp, Artificial Sequence
  primer
cttgctcccaaacagaatt

SEQ ID NO: 20 - DNA, 23 bp, Artificial Sequence
  primer
aggccatgctccatgcagactgc

SEQ ID NO: 21 - DNA, 24 bp, Artificial Sequence
  primer
acaagcttttaaagagtttcttgt

SEQ ID NO: 22 - DNA, 20 bp, Artificial Sequence
  primer
aggcaatgtgttagtacaca

SEQ ID NO: 23 - DNA, 22 bp, Artificial Sequence
  primer
acatgtcttaaggagtacattt

SEQ ID NO: 24 - DNA, 24 bp, Artificial Sequence
  primer
tctctaatttcctggcaaacagtg

SEQ ID NO: 25 - DNA, 19 bp, Artificial Sequence
  primer
ctgtggaaacatttagagg

SEQ ID NO: 26 - DNA, 23 bp, Artificial Sequence
  primer
gagtgatgctatttttagatcct

SEQ ID NO: 27 - DNA, 24 bp, Artificial Sequence
  primer
tgcctttccacactgacaggtact

SEQ ID NO: 28 - DNA, 24 bp, Artificial Sequence
  primer
tctgttcttcattagcattagaga

SEQ ID NO: 29 - DNA, 20 bp, Artificial Sequence
  primer
gtgattaattcttctttcca

SEQ ID NO: 30 - DNA, 23 bp, Artificial Sequence
  primer
actgtctcattagcgtctatctt

SEQ ID NO: 31 - DNA, 20 bp, Artificial Sequence
  primer
gggtgaaatttgcagtcagt

SEQ ID NO: 32 - DNA, 23 bp, Artificial Sequence
  primer
aatataatcccagcccatgtgca

SEQ ID NO: 33 - DNA, 22 bp, Artificial Sequence
  primer
attgccaaatgcaacctmtgtc

SEQ ID NO: 34 - DNA, 22 bp, Artificial Sequence
  primer
ttggaggaatgagtagggcatt

SEQ ID NO: 35 - DNA, 23 bp, Artificial Sequence
  primer
acagggaacataaactctgatcc

SEQ ID NO: 36 - DNA, 22 bp, Artificial Sequence
  primer
caacacaccaggcaccttcaga

SEQ ID NO: 37 - DNA, 19 bp, Artificial Sequence
  primer
gtttgggaatgcgtgtttt

SEQ ID NO: 38 - DNA, 24 bp, Artificial Sequence
  primer
agaattagaaaatgaaggtagaca

SEQ ID NO: 39 - DNA, 22 bp, Artificial Sequence
  primer
ctcgtagtgcccaggttgatcc

SEQ ID NO: 40 - DNA, 24 bp, Artificial Sequence
  primer
ccacgtacctatcatggtcactgg

SEQ ID NO: 41 - DNA, 23 bp, Artificial Sequence
  primer
ggccaacctctgtaaatctcgtg

SEQ ID NO: 42 - DNA, 23 bp, Artificial Sequence
  primer
ttcaggcccagcaatcttacgtc

SEQ ID NO: 43 - DNA, 24 bp, Artificial Sequence
  primer
ttcccggttgtatatcagctcatg

SEQ ID NO: 44 - DNA, 24 bp, Artificial Sequence
  primer
agaccctgagcttaaacaaatgcc

SEQ ID NO: 45 - DNA, 24 bp, Artificial Sequence
  primer
aataactcagatcttcccagggtg

SEQ ID NO: 46 - DNA, 24 bp, Artificial Sequence
  primer
actcagcaaagggccttatagaag

SEQ ID NO: 47 - DNA, 24 bp, Artificial Sequence
  primer
catttggcaatacagaaacatcag

SEQ ID NO: 48 - DNA, 21 bp, Artificial Sequence
  primer
gcaactgtctgggaatgaggc

SEQ ID NO: 49 - DNA, 24 bp, Artificial Sequence
  primer
tataaacggtattgtccagccttc

SEQ ID NO: 50 - DNA, 24 bp, Artificial Sequence
  primer
atcagcaataccataaccattcag

SEQ ID NO: 51 - DNA, 21 bp, Artificial Sequence
  primer
tctgcttgcacagcccatttg

SEQ ID NO: 52 - DNA, 24 bp, Artificial Sequence
  primer
gctaagcacagttctgggatttgg

SEQ ID NO: 53 - DNA, 24 bp, Artificial Sequence
  primer
tcaacttctctgtcaccataaccc

SEQ ID NO: 54 - DNA, 22 bp, Artificial Sequence
  primer
aacattccaatgctcttccacc

SEQ ID NO: 55 - DNA, 23 bp, Artificial Sequence
  primer
atcccaaacatttcaatccaagg

SEQ ID NO: 56 - DNA, 24 bp, Artificial Sequence
  primer
gcccatgaccagaaactagtaacc

SEQ ID NO: 57 - DNA, 24 bp, Artificial Sequence
  primer
cttatctgaaatgcttgggaccag

SEQ ID NO: 58 - DNA, 20 bp, Artificial Sequence
  primer
gaacctggcgtgaccatcag

SEQ ID NO: 59 - DNA, 23 bp, Artificial Sequence
  primer
ctctgcttccactttcctccttc

SEQ ID NO: 60 - DNA, 23 bp, Artificial Sequence
  primer
cgccatgttatatcagggacttg

SEQ ID NO: 61 - DNA, 23 bp, Artificial Sequence
  primer
gtcccagcctctcttgcaactag

SEQ ID NO: 62 - DNA, 24 bp, Artificial Sequence
  primer
aggagcatgtttgttctttgcatc

SEQ ID NO: 63 - DNA, 23 bp, Artificial Sequence
  primer
gggagtcaaccaattgataggtg

SEQ ID NO: 64 - DNA, 24 bp, Artificial Sequence
  primer
agaatgaggcaggaagaaatgaag

SEQ ID NO: 65 - DNA, 15 bp, Homo sapiens
aaaggtargcctccc

SEQ ID NO: 66 - DNA, 15 bp, Homo sapiens
aggacctkggctaga

SEQ ID NO: 67 - DNA, 15 bp, Homo sapiens
cagggtgyaaattac

SEQ ID NO: 68 - DNA, 15 bp, Homo sapiens
catacacrtcctgaa

SEQ ID NO: 69 - DNA, 15 bp, Homo sapiens
catgaaayttttgtt

SEQ ID NO: 70 - DNA, 15 bp, Homo sapiens
cctgcaaygaaataa

SEQ ID NO: 71 - DNA, 15 bp, Homo sapiens
cttatcaygaagcaa

SEQ ID NO: 72 - DNA, 15 bp, Homo sapiens
gcatctgmagatttt

SEQ ID NO: 73 - DNA, 15 bp, Homo sapiens
aaatgaaragcaaac

SEQ ID NO: 74 - DNA, 22 bp, Artificial Sequence
  primer
ggcaggaccttggctagagctg

SEQ ID NO: 75 - DNA, 22 bp, Artificial Sequence
  primer
cagctctagccaaggtcctgcc

SEQ ID NO: 76 - DNA, 22 bp, Artificial Sequence
  primer
ggcaggacctgggctagagctg

SEQ ID NO: 77 - DNA, 22 bp, Artificial Sequence
  primer
cagctctagcccaggtcctgcc

SEQ ID NO: 78 - DNA, 23 bp, Artificial Sequence
  primer
ggaagaggtaccgaccttggcta

SEQ ID NO: 79 - DNA, 23 bp, Artificial Sequence
  primer
ggaagaggtaccgacctgggcta

SEQ ID NO: 80 - DNA, 24 bp, Artificial Sequence
  primer
gggaagaggtaccgacctgttgta

SEQ ID NO: 81 - DNA, 22 bp, Artificial Sequence
  primer
cgtgttgaccagtcgctagcca

(19) United States
(12) Patent Application Publication
(10) Pub. No.: US 2005/0014173 A1
(43) Pub. Date: Jan. 20, 2005

(54) Parkinson's disease markers

(76) Inventor: Matthew J. Farrer, Jacksonville Beach, FL (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/839,688

(22) Filed: May 5, 2004

Related U.S. Application Data
(60) Provisional application No. 60/468,832, filed on May 8, 2003.

(51) Int. Cl. C07K 14/705; C12Q 1/68; C07H 21/04

(52) U.S. Cl. 435/6; 530/350; 435/69.1; 435/320.1; 435/325; 536/23.5

(57) ABSTRACT

Nucleic acids and polypeptides are provided that are associated with PD. Methods and articles of manufacture for screening individuals for susceptibility to PD, including susceptibility to a specific PD phenotype, are also disclosed.

### CROSS-REFERENCE TO RELATED APPLICATIONS

This application claims the benefit under 35 U.S.C. § 119 (e) of prior provisional application Ser. No. 60/468,832, filed May 8, 2003, incorporated by reference in its entirety herein.

### STATEMENT AS TO FEDERALLY SPONSORED RESEARCH

Funding for the work described herein was provided in part by the federal government, which may have certain rights in the invention.

### TECHNICAL FIELD

This invention relates to Parkin nucleic acid sequence and polypeptide variants, and more particularly to Parkin nucleic acid sequence and polypeptide variants associated with Parkinson's disease.

### BACKGROUND

Parkinson's disease (PD) is the second most common neurodegenerative disorder after Alzheimer's disease, presently affecting over one million people in the United States alone. The disease is characterized by clinical symptoms such as resting tremor, bradykinesia, and rigidity. PD can be manifested as a number of phenotypes, including juvenile-onset (<21 years), early-onset (<45 years), and late-onset disease (>45 years). Deletions, duplications, and point mutations in the gene known as Parkin were first associated with autosomal recessive juvenile parkinsonism (AR-JP), a rare disorder characterized by early onset movement changes similar to the classic clinical symptoms of idiopathic PD. Parkin mutations have also been reported in many cases of idiopathic, clinically diagnosed PD, including up to 49% of early-onset European patients with a family history compatible with recessive inheritance. The Parkin mutation-associated PD phenotypes encompass juvenile-onset, early-onset, and late-onset disease.

Many of the Parkin mutations are present in the open reading frame, and include, for example, point mutations, whole exon and single base pair deletions, exon duplications, and intra-exonic deletions. Homozygous, compound heterozygous, and single heterozygous mutations (affecting only one allele of the Parkin gene) have been reported. The observation of patients with both normal and mutant alleles suggests that haploinsufficiency is a risk factor for the disease or that certain mutations are dominant, conferring dominant-negative or toxic gain of function(s). While a number of mutations in the Parkin gene have been identified, it would be useful to identify additional mutations, particularly those correlated with a particular PD phenotype.

### SUMMARY

The invention is based on the discovery of sequence variants that occur in both coding and non-coding regions of Parkin nucleic acids. Certain Parkin nucleic acid variants occur in coding regions and encode Parkin polypeptides that may exhibit altered activities, e.g., metal binding and/or altered ubiquitination properties, relative to the wild type Parkin protein. Other Parkin nucleic acid variants occur in non-coding regions and may alter regulation of transcription, translation, and/or splicing of the Parkin nucleic acid. Discovery of these sequence variants and their correlation with PD allows individuals to be screened for susceptibility to PD, including susceptibility to a specific PD phenotype.

Accordingly, in one embodiment, the invention provides isolated nucleic acid molecules having a Parkin nucleic acid sequence. The nucleic acid molecules are at least ten nucleotides in length. The Parkin nucleic acid sequence includes a nucleotide sequence variant at a position selected from: position −227, −258, −1511, −2605, −2983, −3030, −3228, −3807, or −4578 relative to the guanine (position +1) of the transcription start site of the Parkin promoter given in SEQ ID NO: 1; position 1326 relative to the T at position +1 of SEQ ID NO:11; position 1422 relative to the T at position +1 of SEQ ID NO:11; position +2 or position +17 relative to the guanine (position +1) in the splice donor site of Intron 5 in SEQ ID NO: 4; position +1 in the splice donor site of Intron 7 within SEQ ID NO:5; position 951 relative to the T at position +1 of SEQ ID NO:11; position 202 relative to the T at position +1 of SEQ ID NO:11; or position 500 relative to the T at position +1 of SEQ ID NO:11. The nucleotide sequence variant can be a nucleotide substitution, nucleotide insertion, or a nucleotide deletion. For example, the nucleotide sequence variant can be a guanine substitution for adenine at position −227 relative to the guanine of the transcription start site of the Parkin promoter given in SEQ ID NO: 1, or a guanine substitution for thymine at position −258 relative to the guanine of the transcription start site of the Parkin promoter given in SEQ ID NO: 1.

In other embodiments, the nucleotide sequence variant can be a thymine substitution for guanine at position 1326 relative to the T at position +1 in SEQ ID NO:11; a cytosine substitution for thymine at position 1422 relative to the T at position +1 in SEQ ID NO:11; an adenine substitution for thymine at the +2 position relative to the guanine in the splice donor site of Intron 5 within SEQ ID NO: 4; a cytosine substitution for guanine at position +1 of the splice donor site of Intron 7 within SEQ ID NO: 5; a cytosine substitution for guanine at position 951 relative to the T at position +1 of SEQ ID NO. 11; a guanine substitution for adenine at position 202 relative to the T at position +1 SEQ ID NO. 11; a cytosine substitution for adenine at position +17 relative to the guanine in the splice donor site of Intron 5 within SEQ ID NO: 4, or a nucleotide insertion of the nucleotides 5′-CCA-3′ after position 500 relative to the T at position +1 of SEQ ID NO:11.

A Parkin nucleic acid sequence can include a sequence variant associated with Parkinson's disease, including autosomal recessive juvenile parkinsonism, early-onset Parkinson's disease, juvenile-onset Parkinson's disease, or late onset Parkinson's disease. For example, one sequence variant associated with late-onset Parkinson's disease is a guanine substitution for thymine at position −258 relative to the guanine of the transcription start site of the Parkin promoter given in SEQ ID NO: 1.

In another aspect, the invention provides isolated nucleic acid molecules encoding Parkin polypeptides, where the polypeptides include a Parkin amino acid sequence variant relative to the amino acid sequence of SEQ ID NO: 9. The amino acid sequence variant can be at residue 34, 284, or 441. For example, the amino acid sequence variant can be an Arg at residue 441; an Arg at residue 34, or an Arg at residue 284. The amino acid sequence variant can include residues 1-408 relative to the amino acid sequence of SEQ ID NO: 9. The amino acid sequence variant can be an insertion of an amino acid after amino acid residue 133 of SEQ ID NO:9. For example, the amino acid sequence variant can be an insertion of a Pro after amino acid residue 133.

It is another object of the invention to provide isolated Parkin polypeptides. The polypeptides can have an amino acid sequence variant relative to the amino acid sequence of SEQ ID NO:9. The amino acid sequence variant can be an Arg at residue 34; an Arg at residue 284; an Arg at residue 441; or an insertion of a proline after amino acid position 133 of SEQ ID NO:9. An activity of the polypeptide can be altered relative to wild type Parkin polypeptide of SEQ ID NO:9.

In another aspect, the invention provides a method for determining the susceptibility of a subject to Parkinson's disease. The method includes providing a nucleic acid sample from the subject and determining if a Parkin nucleotide sequence variant at position −258 relative to the guanine (position +1) of the transcription start site of the Parkin promoter (SEQ ID NO: 1) is present or absent in the nucleic acid sample, where the presence of the nucleotide sequence variant is associated with increased susceptibility of the subject to Parkinson's disease. The subject can be a mammal (e.g., a human), and the nucleic acid sample can be genomic DNA or cDNA. Determining a patient's susceptibility to Parkinson's disease may be performed by contacting the nucleic acid sample with an article of manufacture that includes a substrate, where the substrate includes a plurality of discrete regions and where each of the regions includes a different population of nucleic acid molecules. The nucleic acid molecules are at least 10 nucleotides in length, and at least one population of nucleic acid molecules includes a guanine substitution for thymine at position −258 relative to the guanine (position +1) of the transcription start site of the Parkin promoter given in SEQ ID NO: 1. The method includes determining if the nucleic acid sample is bound to the article of manufacture. In some embodiments, at least one of the populations includes a wild-type Parkin nucleic acid sequence. In other embodiments, the method further includes detecting the presence or absence of one or more additional Parkin nucleotide sequence variants. The one or more additional Parkin nucleotide sequence variants can be at a position selected from: position −227, −1511, −2605, −2983, −3030, −3228, −3807, or −4578 relative to the guanine (position +1) of the transcription start site of the Parkin promoter given in SEQ ID NO: 1; position 1326 relative to the T at position +1 of SEQ ID NO:11; position 1422 relative the T at position +1 of SEQ ID NO:11; position +2 or position +17 relative to the guanine (position +1) in the splice donor site of Intron 5 within SEQ ID NO:4; position +1 in the splice donor site of Intron 7 within SEQ ID NO:5; position 951 relative to the T at position +1 of SEQ ID NO:11; position 202 relative to the T at position +1 of SEQ ID NO:11; or position 500 relative to the T at position +1 of SEQ ID NO:11.

In another aspect, the invention provides a method for diagnosing Parkinson's disease in a subject. The method includes providing a nucleic acid sample from a subject, and determining whether the nucleic acid sample includes a Parkin nucleotide sequence variant at position −258 relative to the guanine (position +1) of the transcription start site of the Parkin promoter given in SEQ ID NO: 1, where the presence of the Parkin nucleotide sequence variant is diagnostic of Parkinson's disease. For example, the Parkin nucleotide sequence variant at position −258 relative to the guanine of the transcription start site of the Parkin promoter can be a guanine substitution for thymine at position −258.

In yet another aspect, isolated nucleic acid molecules having a Parkin nucleic acid sequence are provided. The nucleic acid molecules are at least ten nucleotides in length, and the Parkin nucleic acid sequence includes a nucleotide sequence variant at a position within the Parkin core promoter set forth in SEQ ID NO: 10. The nucleotide sequence variant can be at a position selected from positions −259, −258, −257, −256, −255, −254, or −253 relative to the guanine (position +1) of the transcription start site of the Parkin core promoter given in SEQ ID NO: 10. In some embodiments, the nucleotide sequence variant affects the binding of an NF1-like protein to the isolated nucleic acid. For example, the binding of an NF1-like protein may be reduced relative to binding of the NF1-like protein to a corresponding wild-type Parkin core promoter sequence. The nucleotide sequence variant can also affect the binding of a protein present in human substantia nigra to the isolated nucleic acid. For example, the binding of a protein in human substantia nigra can be reduced relative to binding of the protein to a corresponding wild-type Parkin core-promoter sequence.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 shows the nucleotide sequence of the homo sapiens parkin promoter (SEQ ID NO:1; Accession No. AF350258). The underlined G at position 5119 (5′-GGCCTGGAGG-3′, SEQ ID NO:13), is denoted +1 herein, and the start of transcription of the parkin gene. 5′ nucleotides are counted from the adjacent 5′ A, denoted −1 herein.

FIG. 2 sets forth the sequence of exon 11 and flanking intronic sequence (SEQ ID NO:2). SEQ ID NO:2 shows the G>T mutation (denoted as a K for G or T) at position 1326 relative to the T at position +1 of SEQ ID NO:11 (FIG. 11). A mutation from Glu to a stop codon at amino acid residue 409 of the wild type Parkin protein (SEQ ID NO:9) results.

FIG. 3 sets forth the sequence of exon 12 with flanking intronic sequence (SEQ ID NO:3). SEQ ID NO:3 shows the T>C mutation (denoted as a Y for T or C) at position 1422 relative to the T at position +1 of SEQ ID NO:11 (FIG. 11). A mutation from Cys to Arg at amino acid residue 441 of the wild type Parkin protein (SEQ ID NO:9) results.

FIG. 4 shows the sequence (SEQ ID NO:4) around the intron 5 +2 T>A mutation (denoted as a W for T or A) and around the intron 5 +17 A>C mutation (denoted as an M for an a or C), both relative to the guanine (position +1 and underlined) in the splice donor site of intron 5.

FIG. 5 shows the sequence (SEQ ID NO:5) around the intron 7 +1 G>C mutation (denoted as an S for a G or C), mutating the guanine (position +1) of the splice donor site of intron 7.

FIG. 6 sets forth the sequence of exon 7 and flanking intronic sequence (SEQ ID NO:6). SEQ ID NO:6 shows the G>C mutation (denoted as an S for a G or C) at position 951 relative to the T at position +1 of SEQ ID NO:11. A mutation from Gly to Arg at amino acid residue 284 of the wild type Parkin protein (SEQ ID NO:9) results.

FIG. 7 sets forth the sequence of exon 2 and flanking intronic sequence (SEQ ID NO:7). SEQ ID NO:7 shows the A>G mutation (denoted as an R for A or G) at position 202 relative to the T at position +1 of SEQ ID NO:11 (FIG. 11). A mutation from Gln to Arg at amino acid residue 34 of the wild type Parkin protein (SEQ ID NO:9) results.

FIG. 8 sets forth the sequence of exon 3 and flanking intronic sequence (SEQ ID NO:8), and indicates the insertion of 3 base pairs (denoted as CCA) after position 500 (after position 499) relative to the T at position +1 of SEQ ID NO:11. An in-frame insertion of a proline after amino acid residue 133 of the wild type Parkin protein (SEQ ID NO:9) results.

FIG. 9 is the amino acid sequence of the wild type Parkin protein (SEQ ID NO: 9; Accession No. NP_{—}004553).

FIG. 10 is the Parkin core promoter (SEQ ID NO:10). The G of the transcription start site is position +1. Various transciption factor consensus sequences are indicated. The start codon is indicated with a double underline.

FIG. 11 is the complete Parkin mRNA (SEQ ID NO:11; Accession No. AB009973). By convention with the published literature, the first T is labeled position +1. However, the first 12 bases (5′-tccgggaggatt-3′, SEQ ID NO:14) of SEQ ID NO:11 are incorrect; the correct sequence from the start of transcription is 5′-GGATTTA-3′, as shown in FIG. 12 (SEQ ID NO:12).

FIG. 12 shows the complete (correct) Parkin mRNA sequence (SEQ ID NO:12). Note that 5′-GGATTTA-3′ is the correct initial sequence, with the underlined G as the start of transcription and position +1. Compare FIG. 11 and SEQ ID NO:11.

FIG. 13 shows an electromobility shift assay (EMSA) about the −258 polymorphism using allele-specific probes. Lane 1, no nuclear extract (probe alone); lanes 2-16, 5 μg of human substantia nigra nuclear protein extract. Unlabeled competitor allele-specific probe was added to lanes 3-9 (T allele) and lanes 10-16 (G allele).

### DETAILED DESCRIPTION

The invention features Parkin nucleic acid and polypeptide sequence variants. The Parkin gene has 12 exons spanning 1.53 Mb and encodes a Parkin protein having an E3 ubiquitin protein ligase domain at its N-terminal end (1-76 amino acids) and two RING finger motifs (238-293 and 314-377 amino acids) at its C-terminal end. The E3 ubiquitin protein ligase portion indicates that Parkin may attach to proteins to target them for a variety of cellular destinations, including endosomes, lysosomes, and autophagic vesicles, or to the nucleus. Similarly, RING-finger motifs have been shown to mediate a step in the ubiquitination of proteins destined for degradation by the proteasome. Parkin may therefore act as an intermediate in a ubiquitin pathway, controlling levels of other proteins or itself by regulated degradation. In addition, the RING finger domain of the mouse Parkin homolog (RBCK1) has been shown to function as a transcriptional activator, indicating that the Parkin RING finger domain may also directly regulate gene expression.

As described herein, the association of Parkin variants with PD is indicated by the discovery that certain sequence variants within Parkin are correlated with PD, particularly certain phenotypes of PD. “Associated with PD,” means, with respect to a particular variant, that the variant may be present in both alleles, in one allele, or in combination with one or more other variants to result in a phenotype of PD. Detection of a variant prior to the onset of clinical symptoms of PD can be used to screen individuals for susceptibility to PD. Alternatively, detection of a variant coupled with the display of one or more idiopathic PD symptoms can be used to diagnose PD. Parkin variants can lead to a loss of production of functional protein or result in a gain of toxic function of the protein. Alternatively, the variant may increase or decrease production of the encoded protein (e.g., alter transcription and/or translation level), or may cause production of a protein with a sequence, structure, and/or function that differs from the wild-type protein.

1. Isolated Parkin Nucleic Acid Molecules

The invention features isolated nucleic acids that include a Parkin nucleic acid sequence. The Parkin nucleic acid sequence includes a nucleotide sequence variant and nucleotides flanking the sequence variant. As used herein, the term “nucleic acid” refers to both RNA and DNA, including cDNA, genomic DNA, synthetic (e.g., chemically synthesized) DNA, and DNA containing nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine or 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See Summerton and Weller, Antisense Nucleic Acid Drug Dev. (1997) 7(3):187-195; and Hyrup et al. (1996) Bioorgan. Med. Chem. 4(1):5-23. In addition, the deoxyphosphate backbone can be replaced with, for example, a phosphorothioate or phosphorodithioate backbone, a phosphoroamidite, or an alkyl phosphotriester backbone. The nucleic acid can be double-stranded or single-stranded (i.e., a sense or an antisense single strand).

As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that flank the Parkin gene). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

As described herein, isolated Parkin nucleic acid molecules are at least 10 nucleotides in length. For example, the nucleic acid can be about 10, 10-20 (e.g., 10, 11, 12, 13, 14, 15, 16, 17, 18, 19, or 20 nucleotides in length), 20-50, 50-100 or greater than 100 nucleotides in length (e.g., greater than 150, 200, 250, 300, 350, 400, 450, 500, 750, or 1000 nucleotides in length). The full-length human Parkin transcript contains 12 exons and is 1.53 Mb nucleotides in length. A Parkin nucleic acid molecule therefore is not required to contain all or indeed even any of the coding region of the Parkin gene or all of the exons. For example, a Parkin nucleic acid molecule can contain as little as a single exon or a portion of a single exon (e.g., 10 nucleotides from a single exon). In other embodiments, a Parkin nucleic acid molecule may contain none of the coding regions. For example, a Parkin nucleic acid molecule can contain all or a portion of a Parkin promoter. Five kilobases of a Parkin promoter sequence are set forth in SEQ ID. NO:1. Alternatively, a Parkin nucleic acid sequence as described herein can contain all or a portion of a Parkin core promoter as set forth in SEQ ID. NO:10. As used herein, the “Parkin core promoter” means a region of DNA upstream of Parkin exon 1 capable of transcription activation of Parkin in human neuroblastoma cells. In yet other embodiments, the Parkin nucleic acid can be all or a portion of a Parkin intron sequence. Nucleic acid molecules that are less than full-length can be useful, for example, for diagnostic purposes.

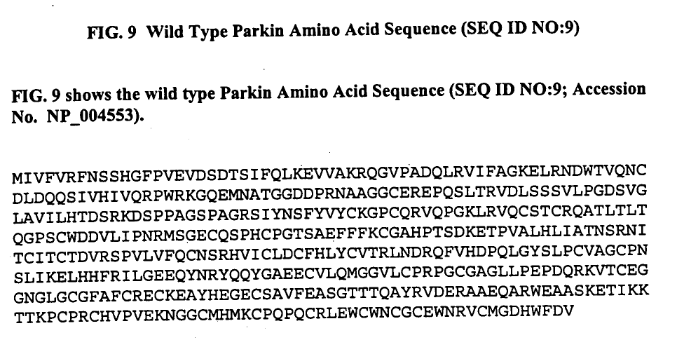

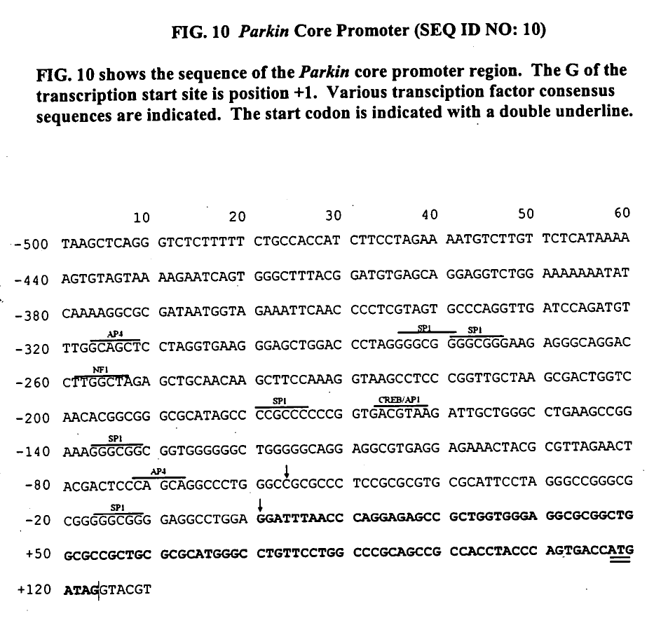

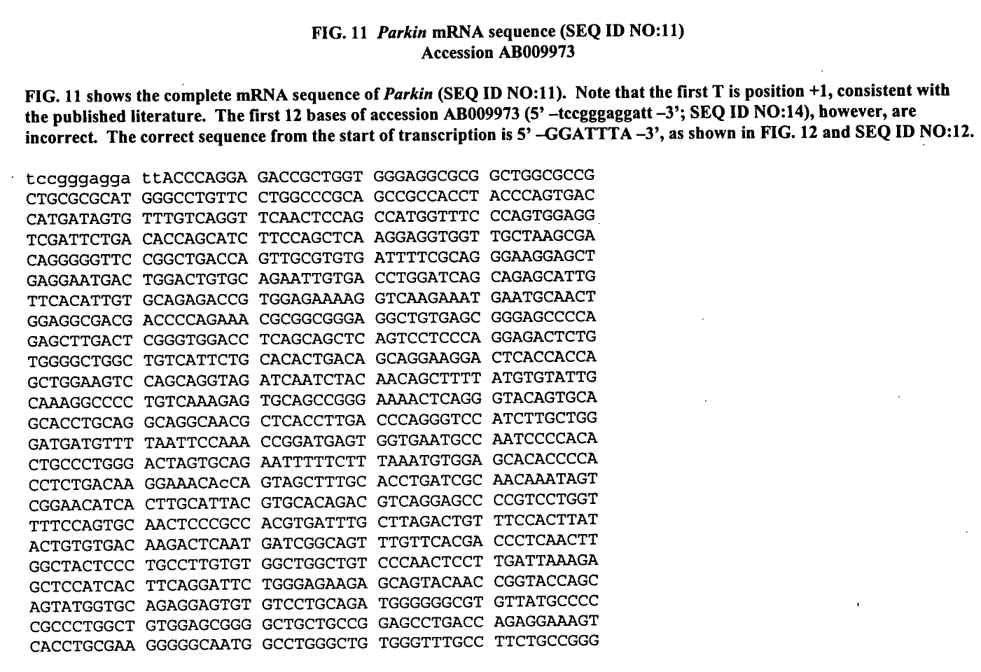

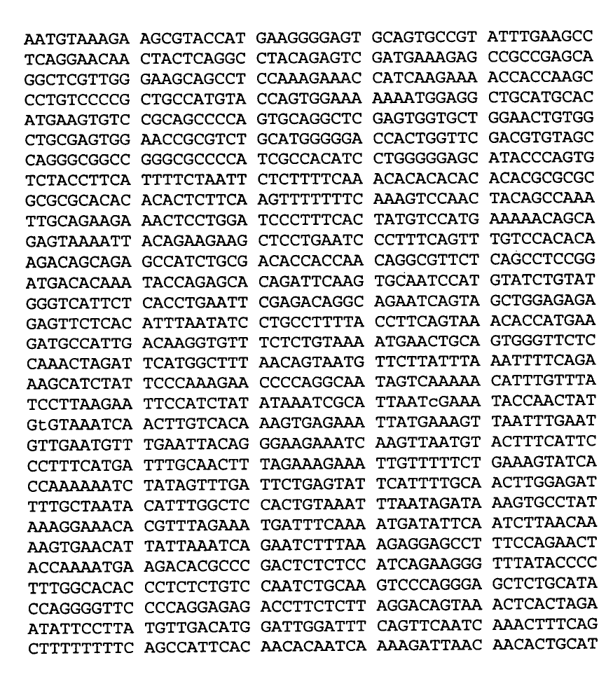

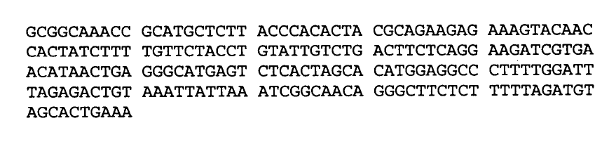

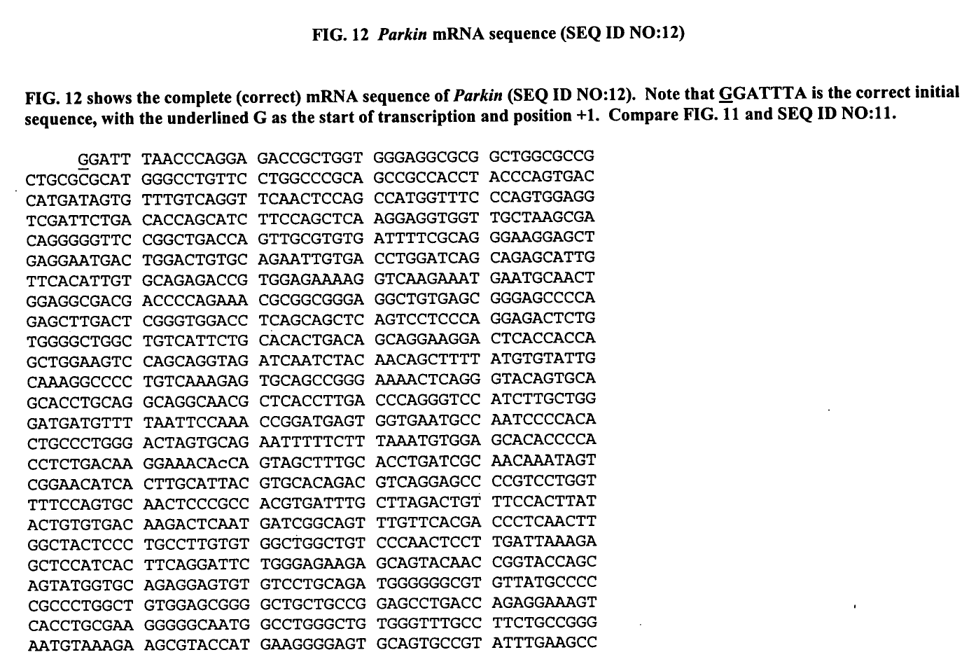

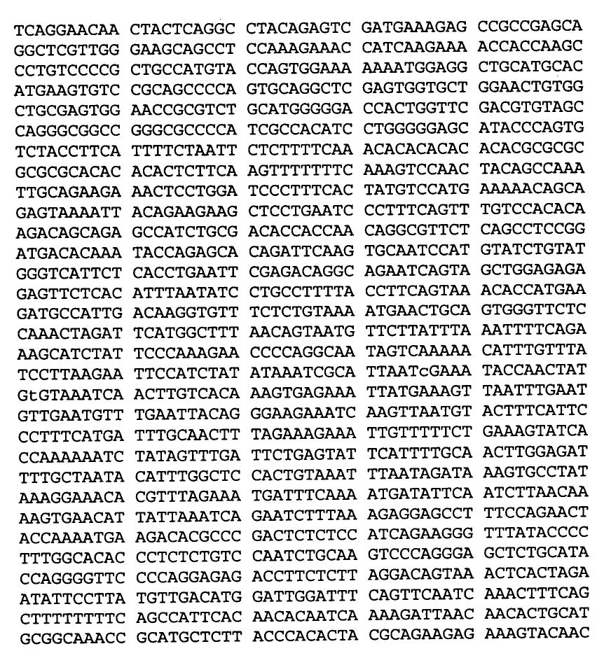

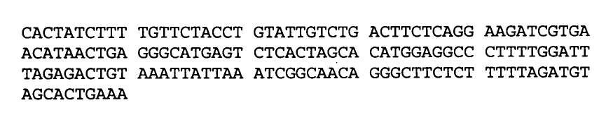

As used herein, “nucleotide sequence variant” refers to any alteration in a Parkin reference sequence, and includes variations that occur in coding and non-coding regions, including exons, introns, promoter regions, and untranslated sequences. Nucleotides are referred to herein by standard one-letter designation (A, C, G, or T), or by the following abbreviations: U=Uracil; R=G or A; Y=T or C; M=A or C; K=G or T; S=G or C; W=A or T; B=G, C, or T; D=A, G, or T; H=A, C, or T; V=A, G, or C; and N=A, G. C, or T. The reference Parkin nucleic acid sequences are provided in SEQ ID NOS:1-8 and in GenBank (Accession No. AF350258 (SEQ ID NO:1)). The reference human Parkin mRNA sequence and individual exons, but not intronic flanking sequences, are provided in FIG. 11 (SEQ ID NO:11; Accession No. AB009973) and in FIG. 12 (SEQ ID NO:12). The reference human Parkin amino acid sequence is provided in FIG. 9 (SEQ ID NO:9; Accession No. NP_{—}004553). The nucleic acid and amino acid reference sequences also are referred to herein as “wild type.”

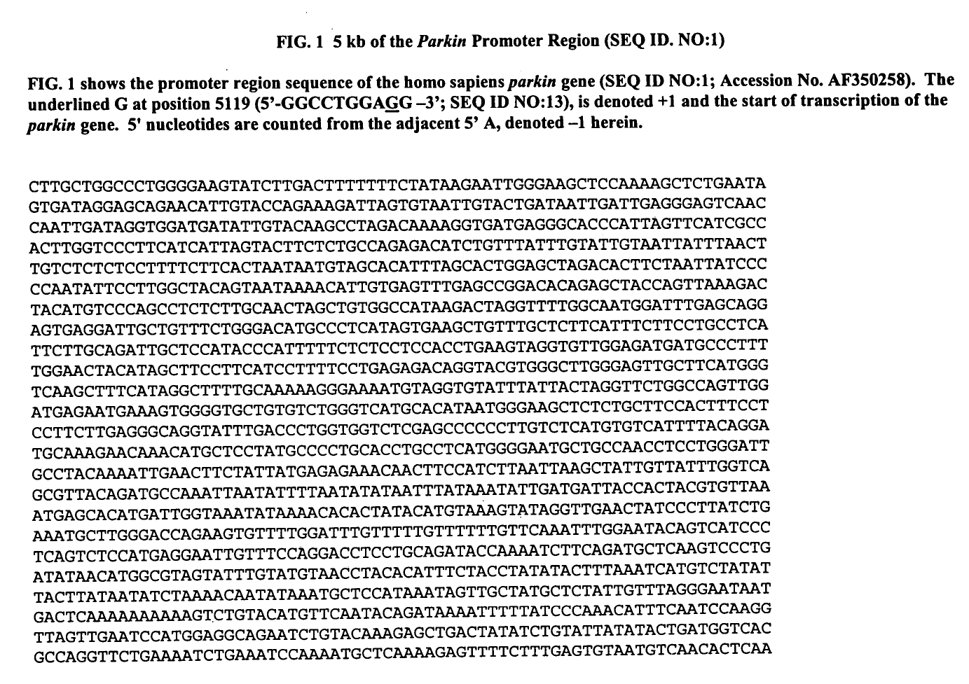

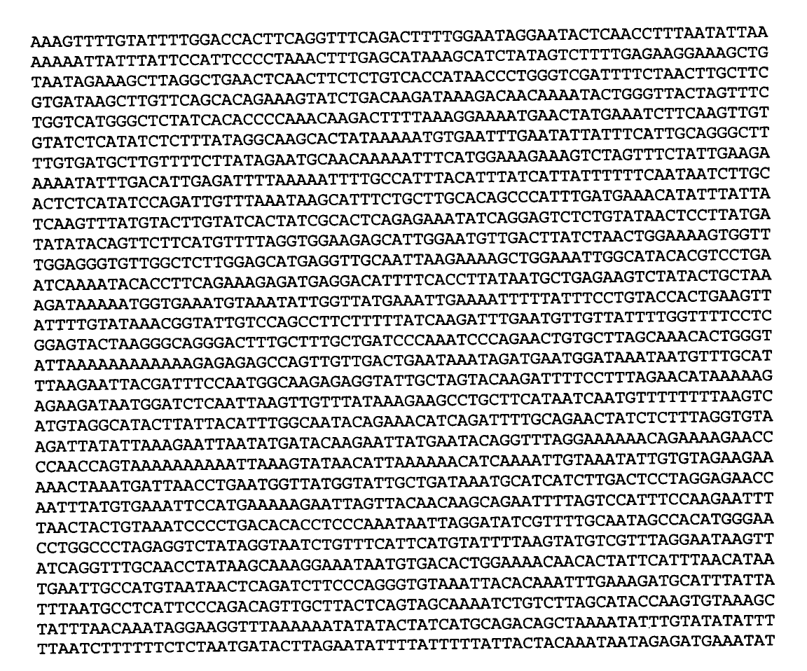

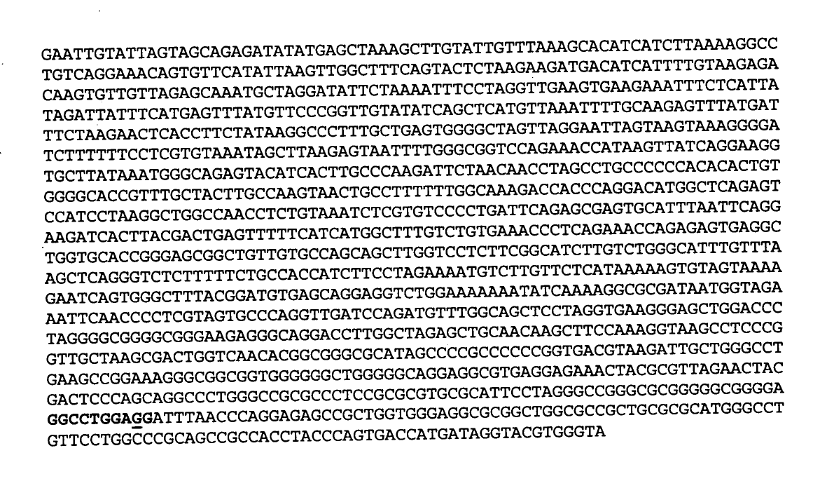

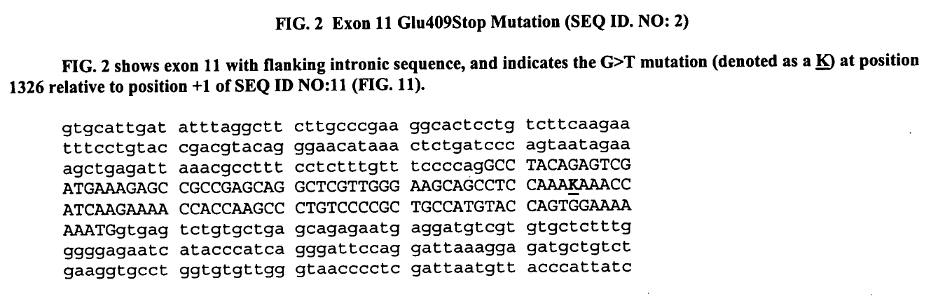

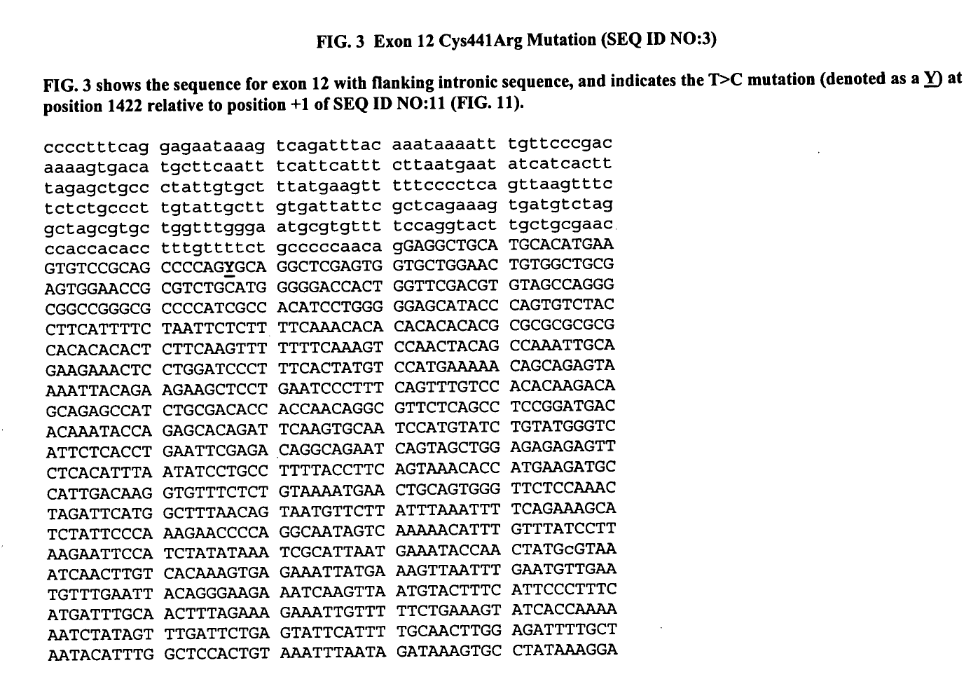

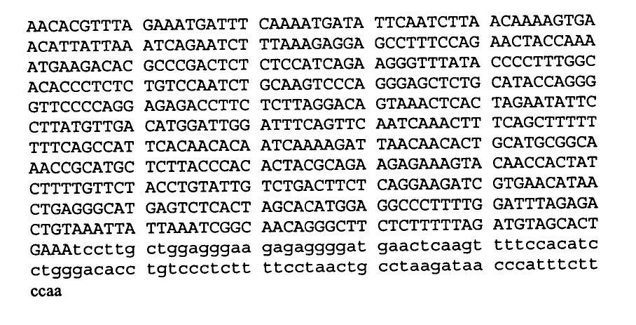

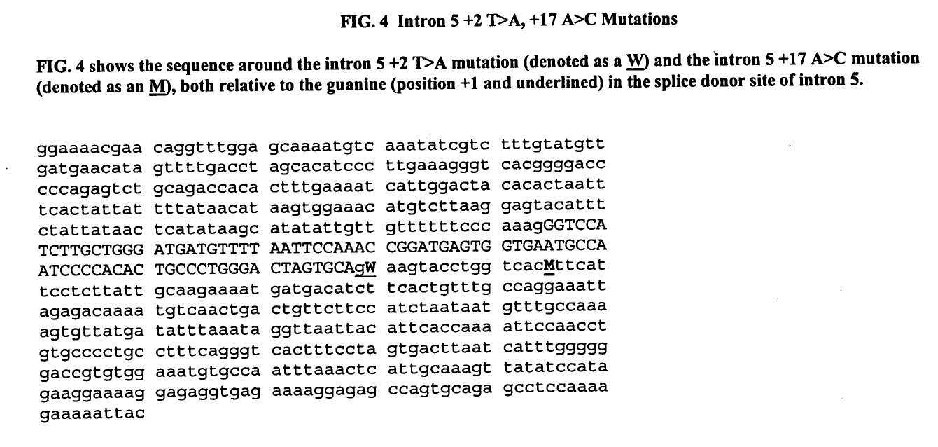

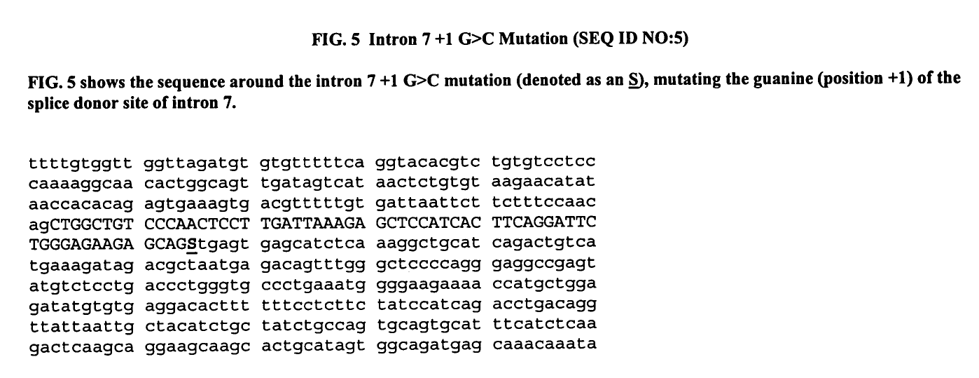

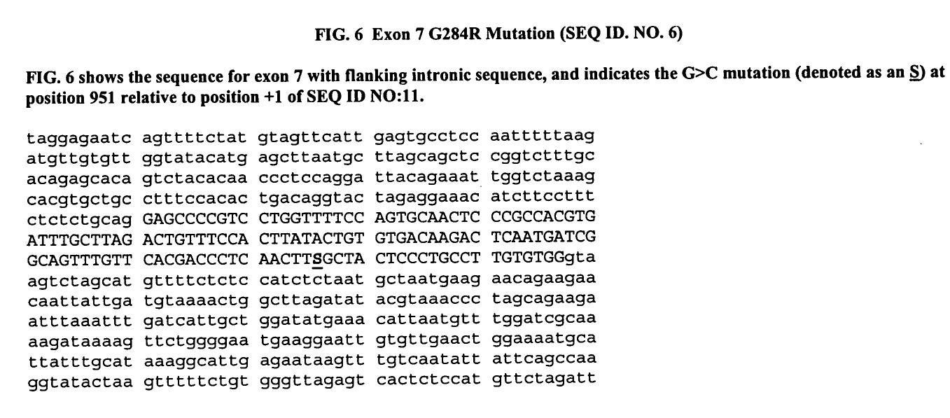

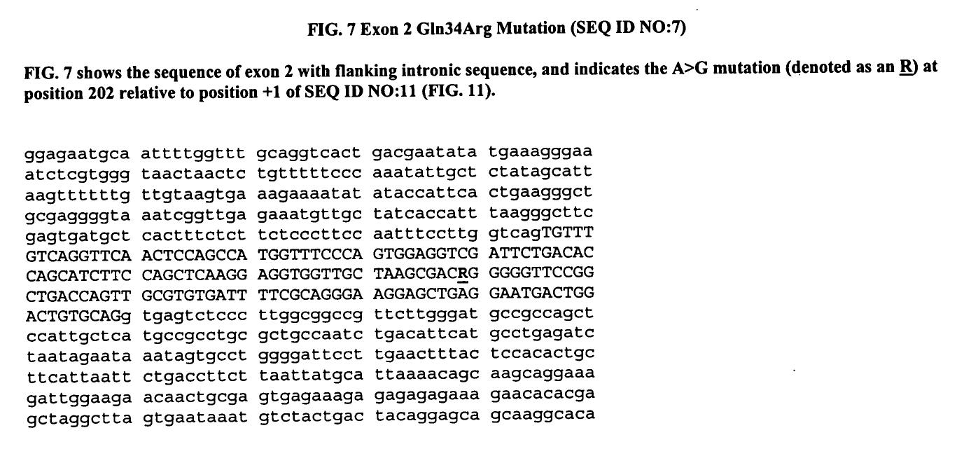

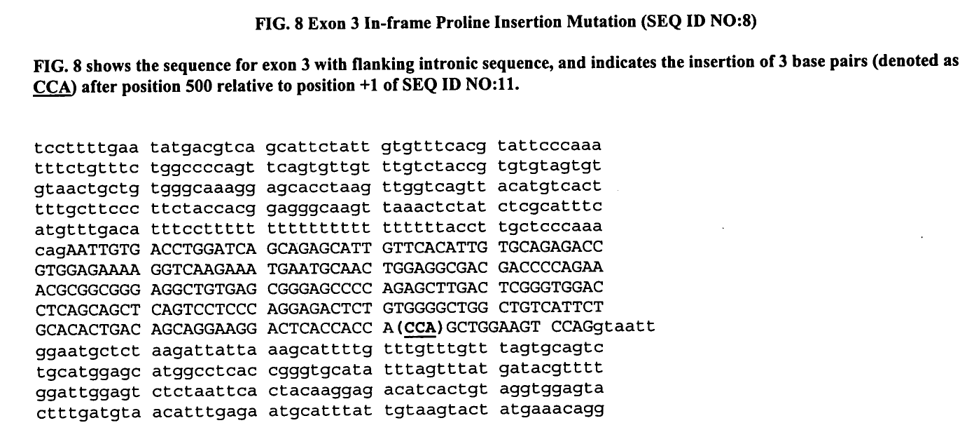

As used herein, positions of nucleotide sequence variants in Parkin promoter sequences are designated as “−X” relative to the “G” (position +1) of the transcription start site. Note that the first position 5′ of G +1 would be labeled “−1,” and not “0.” The G +1 transcription start site is at position 5119 (5′-GGCCTGGAGG, “G +1” underlined; SEQ ID NO:13) of FIG. 1 (SEQ ID NO:1; Accession No. AF350258). To be consistent with published literature, positions of nucleotide sequence variants in Parkin coding sequence are designated as “+X” or “X” relative to the first T at position +1 of FIG. 11 (SEQ ID NO:11; Accession No. AB009973). For example, position 951 relative to the T at position 1 of SEQ ID NO:11 is mutated from a G to a C (as shown in SEQ ID NO: 6), resulting in a mutation in exon 7 of Gly at amino acid residue 284 to an Arg. Although the 5′ end of FIG. 11 (SEQ ID: 11; Accession no. AB009973) is incorrect (see FIG. 12 (SEQ ID NO.12) and West et al. (2001) J. Neurochem. 78:1146-52), this nomenclature is used herein to be consistent with the published literature. Finally, nucleotide sequence variants that occur in introns are designated as “+X” or “X” or as “−X” relative to the “G” (position +1) in the splice donor site (GT).

Sequence variants can be, for example, deletions, insertions, or substitutions at one or more coding nucleotide positions (e.g., 1, 2, 3, 10, or more than 10 positions). Sequence variants that are deletions or insertions can create frame-shifts within the coding region that alter the amino acid sequence of the encoded polypeptide (e.g., mutate the sequence), and thus can affect its structure and function. Alternatively, deletions or insertions within the coding region may be in frame, and can result in the deletion or insertion of amino acids. Isolated nucleic acids can contain, by way of example and not limitation, an insertion after nucleotide position 500 relative to position +1 of SEQ ID NO:11 (shown also in SEQ ID NO:8). The insertion may be, for example, the trinucleotide 5′-CCA-3′, which results in an ‘in-frame’ proline amino acid insertion after amino acid 133 of the wild type Parkin protein. Wild-type, full length Parkin has 465 amino acids but would become 466 amino acids in size. While not being limited by any theory, the insertion of a proline is likely to have deleterious consequences on Parkin function/stability, as a proline generally induces beta-hairpin turns within a protein's secondary structure.

Substitutions include silent mutations that do not affect the amino acid sequence of the encoded polypeptide, missense mutations that alter the amino acid sequence of the encoded polypeptide, and nonsense mutations that prematurely terminate and therefore truncate the encoded polypeptide. Parkin polypeptides, irrespective of length, that differ in amino acid sequence are herein referred to as Parkin polypeptide variants, or variant Parkin polypeptides. The term “polypeptide” refers to a chain of at least four amino acid residues (e.g., 4-8,9-12, 13-15, 16-18, 19-21, 22-100, 100-150, 150-200, 200-300, 300-465 residues, or a full-length Parkin polypeptide). For example, Parkin nucleic acid sequence variants that result in Parkin polypeptide variants include the following missense mutations: a cytosine at position 1422 relative to +1 of SEQ ID NO:11 (see also SEQ ID NO:3) encodes an Arg at position 441 in place of a Cys (Exon 12 Cys441Arg); a cytosine at position 951 relative to position +1 of SEQ ID NO:11 (see also SEQ ID NO:6) encodes an Arg at position 284 in place of a Gly (Exon 7 Gly284Arg); and a guanine at position 202 relative to position +1 of SEQ ID NO:11 (see also SEQ ID NO: 7) encodes an Arg at position 34 in place of a Gln (Exon 2 Gln34Arg). An example of a nonsense mutation includes a thymine at position 1326 relative to position +1 of SEQ ID NO:11 (see also SEQ ID NO:2), thereby encoding a stop codon in place of a Glu at position 409 and resulting in a Parkin polypeptide (Exon 11 Glu409Stop) variant consisting of residues 1-408 of the reference Parkin polypeptide. Variant Parkin polypeptides may or may not have Parkin activity, or may have altered activity (e.g., enhanced or depressed) relative to the reference Parkin polypeptide. Polypeptides that do not have activity or have altered activity are useful for diagnostic purposes (e.g., for producing antibodies having specific binding affinity for variant Parkin polypeptides).

Deletion, insertion, and substitution sequence variants can create or destroy splice sites and thus alter the splicing of a Parkin transcript, such that the encoded polypeptide may contain a deletion or insertion relative to corresponding wild-type polypeptide sequence set forth in SEQ ID NO:9. Sequence variants that affect splice sites of Parkin nucleic acid molecules can result in Parkin polypeptides that lack the amino acids encoded by, for example, exon 5 or portions thereof, or exon 8 or portions thereof. For example, a T substituted for an A at the +2 position relative to the guanine in the splice donor site of intron 5 within SEQ ID NO:4 may affect exon 5 splicing to produce an in-frame truncated transcript. A cytosine at position +17 relative to the guanine in the splice donor site of intron 5 within SEQ ID NO:4 may also lead to exon 5 deletion. For example, deleterious +16 intron splice mutations affect exon 10 inclusion in the tau gene (See Grover, A. et al., J. Biol. Chem., (1999) 274:15134-43). A cytosine at position +1 in splice donor site of intron 7 in SEQ ID NO:5 may lead to an exon 8 deletion and a frame shift (see Rawal N., et al. Neurology (2003) 60:1378-81).

Certain Parkin nucleotide sequence variants may not alter the amino acid sequence. Such variants, however, could alter regulation of transcription as well as mRNA stability. Parkin variants can occur in intron sequences, for example, within introns 1, 2, 3, 4, 5, 6, 7, 8, 9, 10, or 11. In particular, the nucleotide sequence variant can include an adenine substitution at nucleotide +2, or a cytosine substitution at nucleotide +17, both relative to the guanine of the splice donor site, of intron 5 (SEQ ID NO: 4). Intron 7 variants can include a cytosine substitution at nucleotide position +1 of the splice donor site (SEQ ID NO:5; and see Rawal N., et al. Neurology (2003) 60:1378-81).

Alternatively, Parkin nucleotide sequence variants that do not alter the amino acid sequence can occur in the Parkin promoter region set forth in SEQ ID NO:1. Such promoter sequence variants can affect, e.g., reduce or enhance, the binding of proteins, such as DNA-binding transcription factors, relative to the binding of such proteins to a wild type promoter sequence. Such reduced or enhanced binding may affect the rate or amount of transcription of Parkin and/or affect Parkin expression (e.g., in the substantia nigra). For example, the nucleotide sequence of SEQ ID NO:1 can have a guanine at nucleotide −227, a guanine at nucleotide −258, a cytosine at nucleotide −1511, a guanine at nucleotide −2605, a cytosine at nucleotide −2983, a cytosine at nucleotide −3030, a thymine at nucleotide −3228, an adenine at nucleotide −3807, or an adenine at nucleotide −4578, or combinations thereof, where all positions are relative to the guanine (position +1) of the transcription start site of SEQ ID NO:1.

In some embodiments, nucleic acid molecules of the invention can have at least 97% (e.g., 97.5%, 98%, 98.5%, 99.0%, 99.5%, 99.6%, 99.7%, 99.8%, 99.9%, or 100%) sequence identity with a region of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:10, or SEQ ID NO:12 that includes one or more variants described herein. The region of SEQ ID NO:1, 2, 3, 4, 5, 6, 7, 8, 10, or 12 is at least ten nucleotides in length (e.g., ten, 15, 20, 50, 60, 70, 75, 100, 150 or more nucleotides in length). For example, a nucleic acid molecule can have at least 99% identity with a region of SEQ ID NO:1 containing nucleotides −300 to −200 relative to the guanine (position +1) of the Parkin transcription start site, where the nucleotide sequence of SEQ ID NO:1 includes one or more of the variants described herein. For example, the nucleotide sequence of SEQ ID NO:1 can have a guanine at nucleotide −227 or a guanine at nucleotide −258, or both.

In another embodiment, a nucleic acid molecule can have at least 99% identity with a region of SEQ ID NO:2, where the nucleotide sequence of SEQ ID NO:2 includes one or more of the variants described herein. In another embodiment, a nucleic acid molecule can have at least 99% identity with a region of SEQ ID NO:3, where the nucleotide sequence of SEQ ID NO:3 includes one or more of the variants described herein.

A nucleic acid molecule also can have at least 99% identity with a region of SEQ ID NO:4 containing nucleotides −1 to +99 relative to the guanine in the splice donor site of intron 5, where the nucleotide sequence of SEQ ID NO:4 includes one or more of the variants described herein. For example, the nucleotide sequence of SEQ ID NO:4 can have a adenine at nucleotide position +2 or cytosine at position +17 relative to the guanine in the splice donor site of intron 5, and a combination thereof. In another embodiment, a nucleic acid molecule can have at least 99% identity with a region of SEQ ID NO:5 containing nucleotides −20 to +80 relative to the guanine in the splice donor site of intron 7 within SEQ ID NO:5, where the nucleotide sequence of SEQ ID NO:5 includes one or more of the variants described herein. For example, the nucleotide sequence of SEQ ID NO:5 can have a cytosine at position +1 in the splice donor site of intron 7.

In another embodiment, a nucleic acid molecule can have at least 99% identity with a region of SEQ ID NO:6, where the nucleotide sequence of SEQ ID NO:6 includes one or more of the variants described herein. In yet another embodiment, a nucleic acid molecule can have at least 99% identity with a region of SEQ ID NO:7, where the nucleotide sequence of SEQ ID NO:7 includes one or more of the variants described herein. In still another embodiment, a nucleic acid molecule can have at least 99% identity with a region of SEQ ID NO:8, where the nucleotide sequence of SEQ ID NO:8 includes one or more of the variants described herein.

Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from Fish & Richardson's web site (www.fr.com/blast) or the U.S. government's National Center for Biotechnology Information web site (www.ncbi.nlm.nih.gov). Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. The following command will generate an output file containing a comparison between two sequences: C:\Bl2seq-i c:\seq1.txt-j c:\seq2.txt-p blastn-o c:\output.txt-q-1-r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a 1000 nucleotide target sequence is compared to the sequence set forth in SEQ ID NO:1, (2) the Bl2seq program presents 969 nucleotides from the target sequence aligned with a region of the sequence set forth in SEQ ID NO: 1 where the first and last nucleotides of that 969 nucleotide region are matches, and (3) the number of matches over those 969 aligned nucleotides is 900, then the 1000 nucleotide target sequence contains a length of 969 and a percent identity over that length of 93 (i.e., 900÷969×100=93).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Isolated nucleic acid molecules of the invention can be produced by standard techniques, including, without limitation, common molecular cloning and chemical nucleic acid synthesis techniques. For example, polymerase chain reaction (PCR) techniques can be used to obtain an isolated nucleic acid containing a Parkin nucleotide sequence variant. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence. replication, or nucleic acid sequence-based amplification also can be used to obtain isolated nucleic acids. See, for example, Lewis Genetic Engineering News, 12(9):1 (1992); Guatelli et al., Proc. Natl. Acad. Sci. USA, 87:1874-1878 (1990); and Weiss, Science, 254:1292 (1991).

Isolated nucleic acids of the invention also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector if desired.

Isolated nucleic acids of the invention also can be obtained by mutagenesis. For example, the reference sequences set forth in SEQ ID NOs:1-8 and SEQ ID NOs:10-12 can be mutated using standard techniques including oligonucleotide-directed mutagenesis and site-directed mutagenesis through PCR. See Short Protocols in Molecular Biology, Chapter 8, Green Publishing Associates and John Wiley & Sons, edited by Ausubel et al., 1992. Examples of positions that can be modified are described above.

Certain sequence variants described herein are associated with PD. Such sequence variants can result in a change in the encoded polypeptide that can have an effect on the function or activity of the polypeptide, or can result in a change in expression levels of the encoded polypeptide. These changes can include, for example, a truncation, a frame-shifting alteration, a substitution at a highly conserved position, or a substitution in the Parkin promoter. Conserved positions can be identified by inspection of a nucleotide or amino acid sequence alignment showing related nucleic acids or polypeptides from different species. With respect to SEQ ID NO:1, sequence variants that can be associated with PD include, for example, at guanine substitution for thymine at position −258 relative to the guanine of the transcription start site of the Parkin promoter given in SEQ ID NO:1. In particular, this sequence variant is associated with late-onset PD.

In some PD patients, a PD-associated sequence variant can be found on one or both alleles. In other patients, a combination of PD-associated sequence variants can be found on separate alleles of a Parkin gene.

2. Parkin Polypeptides

The invention provides purified Parkin polypeptide variants that are encoded by the Parkin nucleic acid molecules of the invention. A “polypeptide” refers to a chain of at least 10 amino acid residues (e.g., 10, 20, 50, 75, 100, 200, or more than 200 residues), regardless of post-translational modification (e.g., phosphorylation or glycosylation). Typically, a Parkin polypeptide variant of the invention is capable of eliciting a Parkin-specific antibody response (i.e., is able to act as an immunogen that induces the production of antibodies capable of specific binding to the Parkin variant).

A Parkin polypeptide variant can have an amino acid sequence that can include an amino acid sequence variant relative to the wild type reference sequence set forth in SEQ ID NO.9. As used herein, an amino acid sequence variant refers to a deletion, insertion, or substitution at one or more amino acid positions (e.g., 1, 2, 3, 10, or more than 10 positions). For example, an isolated Parkin polypeptide variant can have an amino acid sequence substitution variant at one or more of amino acid residues 34, 284, or 441. In particular, an Arg can be substituted at residue 34; an Arg can be substituted at residue 284; or an Arg can be substituted at residue 441. Alternatively, an isolated Parkin polypeptide variant can have an amino acid insertion sequence variant of a Pro after position 133. A Parkin polypeptide variant may have one or more additional sequence variants in addition to the variants described previously, provided that the polypeptide has an amino acid sequence that is at least 80% identical (e.g., 80%, 85%, 90%, 95%, or 99% identical) over its length to the sequence set forth in SEQ ID NO:9.

Percent sequence identity is calculated by determining the number of matched positions in aligned amino acid sequences, dividing the number of matched positions by the total number of aligned amino acids, and multiplying by 100. The percent identity between amino acid sequences therefore is calculated in a manner analogous to the method for calculating the identity between nucleic acid sequences, using the Bl2seq program from the stand-alone version of BLASTZ containing BLASTN version 2.0.14 and BLASTP version 2.0.14; see subsection 1, above. A matched position refers to a position in which identical residues occur at the same position in aligned amino acid sequences. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. The following command will generate an output file containing a comparison between two amino acid sequences: C:\Bl2seq-i c:\seq1.txt-j c:\seq2.txt-p blastp-o c:\output.txt. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive amino acid residues from the target sequence presented in alignment with sequence from the identified sequence starting with any matched position and ending with any other matched position. A matched position is any position where an identical amino acid residue is presented in both the target and identified sequence. Gaps presented in the target sequence are not counted since gaps are not amino acid residues. Likewise, gaps presented in the identified sequence are not counted since target sequence amino acid residues are counted, not amino acid residues from the identified sequence.

The percent identity over a particular length is determined by counting the number of matched positions over that length and dividing that number by the length followed by multiplying the resulting value by 100. For example, if (1) a 1000 amino acid target sequence is compared to the sequence set forth in SEQ ID NO:9, (2) the Bl2seq program presents 200 amino acids from the target sequence aligned with a region of the sequence set forth in SEQ ID NO:9 where the first and last amino acids of that 200 amino acid region are matches, and (3) the number of matches over those 200 aligned amino acids is 180, then the 1000 amino acid target sequence contains a length of 200 and a percent identity over that length of 90 (i.e. 180÷200×100=90). As described for aligned nucleic acids in subsection 1, different regions within a single amino acid target sequence that aligns with an identified sequence can each have their own percent identity. It also is noted that the percent identity value is rounded to the nearest tenth, and the length value will always be an integer.

The deletion, substitution, or insertion of amino acids from a Parkin polypeptide can significantly affect the structure and activity of the variant polypeptide. A deletion can result in a Parkin polypeptide variant that is truncated, for example, after the lysine amino acid at position 408 of SEQ ID NO:9. Amino acids may also be deleted from a Parkin polypeptide as a result of altered splicing (see above).

Amino acid substitutions may be conservative or non-conservative. Conservative amino acid substitutions replace an amino acid with an amino acid of the same class, whereas non-conservative amino acid substitutions replace an amino acid with an amino acid of a different class. Conservative amino acid substitutions typically have little effect on the structure or function of a polypeptide. Examples of conservative substitutions include amino acid substitutions within the following groups: glycine and alanine; valine, isoleucine, and leucine; aspartic acid and glutamic acid; asparagine, glutamine, serine, and threonine; lysine, histidine, and arginine; and phenylalanine and tyrosine.

Non-conservative substitutions may result in a substantial change in the hydrophobicity of the polypeptide or in the bulk of a residue side chain. In addition, non-conservative substitutions may make a substantial change in the charge of the polypeptide, such as reducing electropositive charges or introducing electronegative charges. Examples of non-conservative substitutions include a basic amino acid for a non-polar amino acid, or a polar amino acid for an acidic amino acid. Non-conservative substitutions within a Parkin polypeptide can include, for example, Arg substituted for Cys at amino acid position 441 of SEQ ID NO:9, Arg substituted for Gly at amino acid position 284 of SEQ ID NO:9, and Arg substituted for Gln at amino acid position 34 of SEQ ID NO:9.

The term “purified” as used herein with reference to a polypeptide refers to a polypeptide that either has no naturally occurring counterpart (e.g., a peptidomimetic), has been chemically synthesized and is thus uncontaminated by other polypeptides, or has been separated or purified from other cellular components by which it is naturally accompanied (e.g., other cellular proteins, polynucleotides, or cellular components). Typically, the polypeptide is considered “purified” when it is at least 70% (e.g., 70%, 80%, 90%, 95%, or 99%), by dry weight, free from the proteins and naturally occurring organic molecules with which it naturally associates.

Parkin polypeptides typically contain multiple functional domains (e.g., two or more regions that are responsible for a specific function of the polypeptide.) A Parkin polypeptide may contain one or more ring (RING) finger domains. A RING finger domain can be located, for example, between amino acid residues 238 and 293, or between amino acid residues 314 and 377 of SEQ ID NO:9. If the Parkin polypeptide contains two or more RING finger domains, it may contain an in-between-ring-finger (IBR) domain. A Parkin polypeptide also may include an E3 ubiquitin protein ligase domain. Such a domain may be located between amino acid residues 1 and 76 of SEQ ID NO:9.

In some embodiments, an activity of a Parkin polypeptide variant is altered relative to the reference Parkin polypeptide. The activity can be reduced or enhanced, or the activity may be a different activity. Activity of the Parkin polypeptide variants can be assessed in vitro. For example, the zinc metal binding affinity of a RING finger domain of a Parkin polypeptide variant can be assessed and compared to the wild type zinc binding affinity. Alternatively, E3 ubiquitin ligase activity can be measured directly using HA-tagged ubiquitin either: 1) in vitro with recombinant protein (Parkin (E3 ligase), E2 cofactors (UbcH7), HA-ubiquitin, ATP and substrate (e.g. Pael-R, Cyclin E); or 2) in vivo using cells transfected with wild-type or mutant Parkin and HA-tagged ubiquitin constructs.

Parkin polypeptide variants can be produced by a number of methods, many of which are well known in the art. By way of example and not limitation, Parkin polypeptide variants can be obtained by extraction from a natural source (e.g., from isolated cells, tissues or bodily fluids), by expression of a recombinant nucleic acid encoding the polypeptide, or by chemical synthesis.

Parkin polypeptide variants of the invention can be produced by, for example, standard recombinant technology, using expression vectors encoding Parkin polypeptides. The resulting Parkin polypeptide variants then can be purified. Expression systems that can be used for small or large scale production of Parkin polypeptide variants include, without limitation, microorganisms such as bacteria (e.g., E. coli and B. subtilis) transformed with recombinant bacteriophage DNA, plasmid DNA, or cosmid DNA expression vectors containing the nucleic acid molecules of the invention; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules of the invention; plant cell systems infected with recombinant virus expression vectors (e.g., tobacco mosaic virus) or transformed with recombinant plasmid expression vectors (e.g., Ti plasmid) containing the nucleic acid molecules of the invention; or mammalian cell systems (e.g., primary cells or immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids of the invention.

Suitable methods for purifying the polypeptides of the invention can include, for example, affinity chromatography, immunoprecipitation, size exclusion chromatography, and ion exchange chromatography. See, for example, Flohe et al. (1970) Biochim. Biophys. Acta. 220:469-476, or Tilgmann et al. (1990) FEBS 264:95-99. The extent of purification can be measured by any appropriate method, including but not limited to: column chromatography, polyacrylamide gel electrophoresis, or high-performance liquid chromatography. Variant Parkin polypeptides also can be “engineered” to contain a tag sequence described herein that allows the polypeptide to be purified (e.g., captured onto an affinity matrix). Finally, immunoaffinity chromatography also can be used to purify variant Parkin polypeptides.

The invention also provides antibodies having specific binding activity for Parkin polypeptide variants. Such antibodies can be useful for diagnostic purposes (e.g., an antibody that recognizes a specific Parkin variant could be used to diagnose PD). An “antibody” or “antibodies” includes intact molecules as well as fragments thereof that are capable of binding to an epitope of a Parkin polypeptide variant. The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids. The terms “antibody” and “antibodies” include polyclonal antibodies, monoclonal antibodies, humanized or chimeric antibodies, single chain Fv antibody fragments, Fab fragments, and F(ab)₂ fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies are particularly useful.

In general, a Parkin polypeptide variant is produced as described above, i.e., recombinantly, by chemical synthesis, or by purification of the native protein, and then used to immunize animals. Various host animals including, for example, rabbits, chickens, mice, guinea pigs, and rats, can be immunized by injection of the protein of interest. Depending on the host species, adjuvants can be used to increase the immunological response and include Freund's adjuvant (complete and/or incomplete), mineral gels such as aluminum hydroxide, surface-active substances such as lysolecithin, pluronic polyols, polyanions, peptides, oil emulsions, keyhole limpet hemocyanin, and dinitrophenol. Polyclonal antibodies are contained in the sera of the immunized animals. Monoclonal antibodies can be prepared using standard hybridoma technology. In particular, monoclonal antibodies can be obtained by any technique that provides for the production of antibody molecules by continuous cell lines in culture as described, for example, by Kohler et al. (1975) Nature 256:495-497, the human B-cell hybridoma technique of Kosbor et al. (1983) Immunology Today 4:72, and Cote et al. (1983) Proc. Natl. Acad. Sci. USA 80:2026-2030, and the EBV-hybridoma technique of Cole et al., Monoclonal Antibodies and Cancer Therapy, Alan R. Liss, Inc. pp. 77-96 (1983). Such antibodies can be of any immunoglobulin class including IgM, IgG, IgE, IgA, IgD, and any subclass thereof. The hybridoma producing the monoclonal antibodies of the invention can be cultivated in vitro or in vivo.

A chimeric antibody is a molecule in which different portions are derived from different animal species, such as those having a variable region derived from a mouse monoclonal antibody and a human immunoglobulin constant region. Chimeric antibodies can be produced through standard techniques.

Antibody fragments that have specific binding affinity for Parkin polypeptide variants can be generated by known techniques. Such antibody fragments include, but are not limited to, F(ab′)₂ fragments that can be produced by pepsin digestion of an antibody molecule, and Fab fragments that can be generated by deducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

Once produced, antibodies or fragments thereof can be tested for recognition of a Parkin polypeptide variant by standard immunoassay methods including, for example, enzyme-linked immunosorbent assay (ELISA) or radioimmuno assay (RIA). See, Short Protocols in Molecular Biology, eds. Ausubel et al., Green Publishing Associates and John Wiley & Sons (1992).

Suitable antibodies typically have equal binding affinities for recombinant and native proteins.

3. Vectors and Host Cells

The invention also provides vectors containing Parkin nucleic acids such as those described above. As used herein, a “vector” is a replicon, such as a plasmid, phage, or cosmid, into which another DNA segment may be inserted so as to bring about the replication of the inserted segment. The vectors of the invention can be expression vectors. An “expression vector” is a vector that includes one or more expression control sequences, and an “expression control sequence” is a DNA sequence that controls and regulates the transcription and/or translation of another DNA sequence.

In the expression vectors of the invention, the nucleic acid is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin (HA), or Flag™ tag (Kodak, New Haven, Conn.) sequences typically are expressed as a fusion with the encoded polypeptide. Such tags can be inserted anywhere within the polypeptide including at either the carboxyl or amino terminus.

The invention also provides host cells containing vectors of the invention. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, New York (1989), and reagents for transformation and/or transfection are commercially available (e.g., Lipofectin (Invitrogen/Life Technologies); Fugene (Roche, Indianapolis, Ind.); and SuperFect (Qiagen, Valencia, Calif.)).

Non-Human Mammals

The invention features non-human mammals that include Parkin nucleic acids of the invention, as well as progeny and cells of such non-human mammals. Non-human mammals include, for example, rodents such as rats, guinea pigs, and mice, and farm animals such as pigs, sheep, goats, horses, and cattle. Non-human mammals of the invention can express a Parkin variant nucleic acid in addition to an endogenous Parkin (e.g., a transgenic non-human that includes a Parkin nucleic acid randomly integrated into the genome of the non-human mammal). Alternatively, an endogenous Parkin nucleic acid can be replaced with a Parkin variant nucleic acid of the invention by homologous recombination. See, Shastry, Mol. Cell Biochem., (1998) 181(1-2):163-179, for a review of gene targeting technology.

In one embodiment, non-human mammals are produced that lack an endogenous Parkin nucleic acid (i.e., a knockout), and then a Parkin variant nucleic acid of the invention is introduced into the knockout non-human mammal. Nucleic acid constructs used for producing knockout non-human mammals can include a nucleic acid sequence encoding a selectable marker, which is generally used to interrupt the targeted exon site by homologous recombination. Typically, the selectable marker is flanked by sequences homologous to the sequences flanking the desired insertion site. It is not necessary for the flanking sequences to be immediately adjacent to the desired insertion site. Suitable markers for positive drug selection include, for example, the aminoglycoside 3N phosphotransferase gene that imparts resistance to geneticin (G418, an aminoglycoside antibiotic), and other antibiotic resistance markers, such as the hygromycin-B-phosphotransferase gene that imparts hygromycin resistance. Other selection systems include negative-selection markers such as the thymidine kinase (TK) gene from herpes simplex virus. Constructs utilizing both positive and negative drug selection also can be used.

For example, a construct can contain the aminoglycoside phosphotransferase gene and the TK gene. In this system, cells are selected that are resistant to G418 and sensitive to gancyclovir.

To create non-human mammals having a particular gene inactivated in all cells, it is necessary to introduce a knockout construct into the germ cells (sperm or eggs, i.e., the “germ line”) of the desired species. Genes or other DNA sequences can be introduced into the pronuclei of fertilized eggs by microinjection. Following pronuclear fusion, the developing embryo may carry the introduced gene in all its somatic and germ cells because the zygote is the mitotic progenitor of all cells in the embryo. Since targeted insertion of a knockout construct is a relatively rare event, it is desirable to generate and screen a large number of animals when employing such an approach. Because of this, it can be advantageous to work with the large cell populations and selection criteria that are characteristic of cultured cell systems. However, for production of knockout animals from an initial population of cultured cells, it is necessary that a cultured cell containing the desired knockout construct be capable of generating a whole animal. This is generally accomplished by placing the cell into a developing embryo environment of some sort.

Cells capable of giving rise to at least several differentiated cell types are “pluripotent.” Pluripotent cells capable of giving rise to all cell types of an embryo, including germ cells, are hereinafter termed “totipotent” cells. Totipotent murine cell lines (embryonic stem, or “ES” cells) have been isolated by culture of cells derived from very young embryos (blastocysts). Such cells are capable, upon incorporation into an embryo, of differentiating into all cell types, including germ cells, and can be employed to generate animals lacking an endogenous Parkin nucleic acid. That is, cultured ES cells can be transformed with a knockout construct and cells selected in which the Parkin gene is inactivated.

Nucleic acid constructs can be introduced into ES cells, for example, by electroporation or other standard technique. Selected cells can be screened for gene targeting events. For example, the polymerase chain reaction (PCR) can be used to confirm the presence of the transgene.

The ES cells further can be characterized to determine the number of targeting events. For example, genomic DNA can be harvested from ES cells and used for Southern analysis. See, for example, Section 9.37-9.52 of Sambrook et al., Molecular Cloning, A Laboratory Manual, second edition, Cold Spring Harbor Press, Plainview; NY, 1989.

To generate a knockout animal, ES cells having at least one inactivated Parkin allele are incorporated into a developing embryo. This can be accomplished through injection into the blastocyst cavity of a murine blastocyst-stage embryo, by injection into a morula-stage embryo, by co-culture of ES cells with a morula-stage embryo, or through fusion of the ES cell with an enucleated zygote. The resulting embryo is raised to sexual maturity and bred in order to obtain animals, whose cells (including germ cells) carry the inactivated Parkin allele. If the original ES cell was heterozygous for the inactivated Parkin allele, several of these animals can be bred with each other in order to generate animals homozygous for the inactivated allele.

Alternatively, direct microinjection of DNA into eggs can be used to avoid the manipulations required to turn a cultured cell into an animal. Fertilized eggs are totipotent, i.e., capable of developing into an adult without further substantive manipulation other than implantation into a surrogate mother. To enhance the probability of homologous recombination when eggs are directly injected with knockout constructs, it is useful to incorporate at least about 8 kb of homologous DNA into the targeting construct. In addition, it is also useful to prepare the knockout constructs from isogenic DNA.

Embryos derived from microinjected eggs can be screened for homologous recombination events in several ways. For example, if the Parkin gene is interrupted by a coding region that produces a detectable (e.g., fluorescent) gene product, then the injected eggs are cultured to the blastocyst stage and analyzed for presence of the indicator polypeptide. Embryos with fluorescing cells, for example, are then implanted into a surrogate mother and allowed to develop to term. Alternatively, injected eggs are allowed to develop and DNA from the resulting pups analyzed by PCR or RT-PCR for evidence of homologous recombination.

Nuclear transplantation also can be used to generate non-human mammals of the invention. For example, fetal fibroblasts can be genetically modified such that they contain an inactivated endogenous Parkin gene and express a Parkin nucleic acid of the invention, and then fused with enucleated oocytes. After activation of the oocytes, the eggs are cultured to the blastocyst stage, and implanted into a recipient. See, Cibelli et al., Science, (1998) 280:1256-1258. Adult somatic cells, including, for example, cumulus cells and mammary cells, can be used to produce animals such as mice and sheep, respectively. See, for example, Wakayama et al., Nature, (1998) 394(6691):369-374; and Wilmut et al., Nature, (1997) 385(6619):810-813. Nuclei can be removed from genetically modified adult somatic cells, and transplanted into enucleated oocytes. After activation, the eggs can be cultured to the 2-8 cell stage, or to the blastocyst stage, and implanted into a suitable recipient. Wakayama et al. 1998, supra.

Non-human mammals of the invention such as mice can be used, for example, to screen compounds to treat and/or alleviate the symptoms of PD, e.g., drugs that alter the variant Parkin polypeptide activity. For example, variant Parkin polypeptide activity or toxicity can be assessed in a first group of such non-human mammals in the presence of a compound, and compared with variant Parkin polypeptide activity in a corresponding control group in the absence of the compound. As used herein, suitable compounds include biological macromolecules such as an oligonucleotide (RNA or DNA), or a polypeptide of any length, a chemical compound, a mixture of chemical compounds, or an extract isolated from bacterial, plant, fungal, or animal matter. The concentration of compound to be tested depends on the type of compound and in vitro test data.

Non-human mammals can be exposed to test compounds by any route of administration, including enterally (e.g., orally) and parenterally (e.g., subcutaneously, intravascularly, intramuscularly, or intranasally). Suitable formulations for oral administration can include tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated by methods known in the art. Preparations for oral administration can also be formulated to give controlled release of the compound.

Compounds can be prepared for parenteral administration in liquid form (e.g., solutions, solvents, suspensions, and emulsions) including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives and other additives such as, for example, antimicrobials, anti-oxidants, chelating agents, inert gases, and the like may also be present. Pharmaceutically acceptable carriers for intravenous administration include solutions containing pharmaceutically acceptable salts or sugars. Intranasal preparations can be presented in a liquid form (e.g., nasal drops or aerosols) or as a dry product (e.g., a powder). Both liquid and dry nasal preparations can be administered using a suitable inhalation device. Nebulised aqueous suspensions or solutions can also be prepared with or without a suitable pH and/or tonicity adjustment.

Detecting Parkin Sequence Variants

Methods of the invention can be used to determine whether the Parkin gene of a subject contains a sequence variant or combination of sequence variants, including those identified herein as being associated with PD. Methods of the invention can be used to determine whether both Parkin alleles of a subject contain sequence variants (either the same sequence variant(s) on both alleles or separate sequence variants on each allele), or whether only a single allele of a subject contains sequence variant(s). The identification of one or more PD-associated sequence variants on an allele(s) can be used to determine susceptibility to PD, when clinical symptoms of PD are not present, or to diagnose PD in a patient when clinical symptoms of PD are present. The identification of other sequence variants (e.g., sequence variants not known to be associated with PD) can be used to support a potential diagnosis of PD. The identification of sequence variants on only one allele can serve as an indicator that the subject is a PD carrier.

Parkin nucleotide sequence variants can be detected, for example, by sequencing exons, introns, promoter regions, 5′ untranslated sequences, or 3′ untranslated sequences, by performing allele-specific hybridization, allele-specific restriction digests, mutation specific polymerase chain reactions (MSPCR), by single-stranded conformational polymorphism (SSCP) detection (Schafer et al., 1995, Nat. Biotechnol. 15:33-39), denaturing high performance liquid chromatography (DHPLC, Underhill et al., 1997, Genome Res., 7:996-1005), infrared matrix-assisted laser desorption/ionization (IR-MALDI) mass spectrometry (WO 99/57318), and combinations of such methods.

Genomic DNA generally is used in the analysis of Parkin nucleotide sequence variants. Genomic DNA is typically extracted from a biological sample such as a peripheral blood sample, but can be extracted from other biological samples, including tissues (e.g., mucosal scrapings of the lining of the mouth or from renal or hepatic tissue). Routine methods can be used to extract genomic DNA from a blood or tissue sample, including, for example, phenol extraction. Alternatively, genomic DNA can be extracted with kits such as the QIAamp® Tissue Kit (Qiagen, Chatsworth, Calif.), Wizard® Genomic DNA purification kit (Promega) and the A.S.A.P.™ Genomic DNA isolation kit (Boehringer Mannheim, Indianapolis, Ind.).

Typically, an amplification step is performed before proceeding with the detection method. For example, exons or introns of the Parkin gene can be amplified then directly sequenced. Dye primer sequencing can be used to increase the accuracy of detecting heterozygous samples.

Allele specific hybridization also can be used to detect sequence variants, including complete haplotypes of a mammal. See Stoneking et al., 1991, Am. J. Hum. Genet. 48:370-382; and Prince et al., 2001, Genome Res., 11(1):152-162. In practice, samples of DNA or RNA from one or more mammals can be amplified using pairs of primers and the resulting amplification products can be immobilized on a substrate (e.g., in discrete regions). Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. High stringency conditions can include the use of low ionic strength solutions and high temperatures for washing. For example, nucleic acid molecules can be hybridized at 42° C. in 2×SSC (0.3M NaCl/0.03 M sodium citrate/0.1% sodium dodecyl sulfate (SDS) and washed in 0.1×SSC (0.015M NaCl/0.0015 M sodium citrate), 0.1% SDS at 65° C. Hybridization conditions can be adjusted to account for unique features of the nucleic acid molecule, including length and sequence composition. Probes can be labeled (e.g., fluorescently) to facilitate detection. In some embodiments, one of the primers used in the amplification reaction is biotinylated (e.g., 5′ end of reverse primer) and the resulting biotinylated amplification product is immobilized on an avidin or streptavidin coated substrate.

Allele-specific restriction digests can be performed in the following manner. For nucleotide sequence variants that introduce a restriction site, restriction digest with the particular restriction enzyme can differentiate the alleles. For sequence variants that do not alter a common restriction site, mutagenic primers can be designed that introduce a restriction site when the variant allele is present or when the wild type allele is present. A portion of Parkin nucleic acid can be amplified using the mutagenic primer and a wild type primer, followed by digest with the appropriate restriction endonuclease.

Certain variants, such as insertions or deletions of one or more nucleotides, change the size of the DNA fragment encompassing the variant. The insertion or deletion of nucleotides can be assessed by amplifying the region encompassing the variant and determining the size of the amplified products in comparison with size standards. For example, a region of Parkin can be amplified using a primer set from either side of the variant. One of the primers is typically labeled, for example, with a fluorescent moiety, to facilitate sizing. The amplified products can be electrophoresed through acrylamide gels with a set of size standards that are labeled with a fluorescent moiety that differs from the primer.

PCR conditions and primers can be developed that amplify a product only when the variant allele is present or only when the wild type allele is present (MSPCR or allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction. Patient samples containing solely the wild type allele would have amplification products only in the reaction using the wild type primer. Similarly, patient samples containing solely the variant allele would have amplification products only in the reaction using the variant primer. Allele-specific PCR also can be performed using allele-specific primers that introduce priming sites for two universal energy-transfer-labeled primers (e.g., one primer labeled with a green dye such as fluoroscein and one primer labeled with a red dye such as sulforhodamine). Amplification products can be analyzed for green and red fluorescence in a plate reader. See, Myakishev et al., 2001, Genome 11(1):163-169.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

Alternatively, Parkin variants can be detected by antibodies that have specific binding affinity for variant Parkin polypeptides. Variant Parkin polypeptides and antibodies having specific binding affinity for the same can be produced in various ways, including recombinantly, as discussed above.

Methods for Determining Susceptibility to PD or for Diagnosing PD

The methods of the invention make it possible to determine whether a mammal has a greater susceptibility (e.g., is predisposed) to PD when few or no clinical symptoms are present or obvious. Additional risk factors including, for example, family history and other genetic factors, can be considered when determining susceptibility. Susceptibility to PD can be based on the presence or absence of a single Parkin sequence variant (e.g., position −258 of the Parkin promoter) or based on a variant profile. “Variant profile” refers to the presence or absence of a plurality (i.e., two or more) of Parkin nucleotide sequence variants or Parkin amino acid sequence variants. For example, a variant profile can include the complete Parkin haplotype of the mammal; the presence or absence of a set of common non-synonymous variants (i.e., single nucleotide substitutions that alter the amino acid sequence of a Parking polypeptide); the presence or absence of a set of common variants in the Parkin promoter region; or the presence or absence of a set of common non-synonymous variants and promoter variants. In one embodiment, the variant profile includes detecting the presence or absence of two or more promoter region or non-synonymous variants (e.g., 2, 3, 4 or more variants). In addition, the variant profile can include detecting the presence or absence of any type of Parkin variant together with any other Parkin variant (i.e., a polymorphism pair or groups of polymorphism pairs).

Methods of the invention also allow the diagnosis of PD, typically when coupled with the identification of known clinical symptoms of PD. Diagnosis can be based on the presence or absence of a single Parkin sequence variant (e.g., position −258 of the Parkin promoter) or based on a variant profile, as described above.

Articles of Manufacture

Articles of manufacture of the invention include populations of isolated Parkin nucleic acid molecules or Parkin polypeptides immobilized on a substrate. Suitable substrates provide a base for the immobilization of the nucleic acids or polypeptides, and in some embodiments, allow immobilization of nucleic acids or polypeptides into discrete regions. In embodiments in which the substrate includes a plurality of discrete regions, different populations of isolated nucleic acids or polypeptides can be immobilized in each discrete region. Thus, each discrete region of the substrate can include a different Parkin nucleic acid or Parkin polypeptide sequence variant. Such articles of manufacture can include one or more sequence variants of Parkin, or can include all of the sequence variants known for Parkin. For example, the article of manufacture can include one or more of the sequence variants identified herein, such as the nucleic acid variants that result in amino acid changes of Glu409Stop, Cys441Arg, Gly284Arg, or Gln34Arg, the insertion of Proline after amino acid 133, or the promoter variants identified herein, and one or more other Parkin sequence variants. The article of manufacture can also include a wild type Parkin nucleic acid sequence.

Suitable substrates can be of any shape or form and can be constructed from, for example, glass, silicon, metal, plastic, cellulose, or a composite. For example, a suitable substrate can include a multiwell plate or membrane, a glass slide, a chip, or polystyrene or magnetic beads. Nucleic acid molecules or polypeptides can be synthesized in situ, immobilized directly on the substrate, or immobilized via a linker, including by covalent, ionic, or physical linkage. Linkers for immobilizing nucleic acids and polypeptides, including reversible or cleavable linkers, are known in the art. See, for example, U.S. Pat. No. 5,451,683 and WO98/20019. Immobilized nucleic acid molecules are typically about 20 nucleotides in length, but can vary from about 10 nucleotides to about 1000 nucleotides in length.

In practice, a sample of DNA or RNA from a subject can be amplified, the amplification product hybridized to an article of manufacture containing populations of isolated nucleic acid molecules in discrete regions, and hybridization can be detected. Typically, the amplified product is labeled to facilitate detection of hybridization. See, for example, Hacia et al., Nature Genet., 14:441-447 (1996); and U.S. Pat. Nos. 5,770,722 and 5,733,729.

The invention will be further described in the following examples, which do not limit the scope of the invention described in the claims.

### EXAMPLES

### Example 1

### Detection of Parkin Mutations

A. Patient DNA Material

Twenty patient samples, including subjects from Europe (Lücking et al., “Association between early-onset Parkinson disease and mutations in the Parkin gene,” N. Engl. J. Med. 342:1560-1567 (2000)) and the United States (Farrer M., et al, “Lewy Bodies and Parkinsonism in families with Parkin mutations,” Ann. Neurol. 50:293-300 (2001)) were assessed. Venous whole blood samples were taken and DNA was extracted using standard protocols. All patients met the criteria for PD. Informed consent was obtained from all patients.

B. Exon and Intron Mutation Detection

Point mutations in the Parkin gene were identified or confirmed by direct sequencing.

All twelve coding exons and intron-exon boundaries were examined as described in Farrer et al., “Lewy Bodies and Parkinsonism in families with Parkin mutations,” Ann. Neurol. 50:293-300 (2001). In addition, semi-quantitative multiplex PCR was used for the detection of exon rearrangements (deletions and duplications). Hex-tagged, fluorescently labeled forward primers for Parkin exons were optimized in pooled sets of 2-4 primer pairs for multiplexing along with an internal control. See Table 1, entitled “Mutation Detection Primers for Parkin Gene Analysis.” PCR amplification in the log linear range allowed quantitative assessment of the product. The conditions for the PCR were 80 ng of genomic DNA, 1U Taq polymerase, 5 μL Q solution (Qiagen), 2.5 μL 10× buffer, 5 mM of each dNTP. Initial 95° C. denaturing (5 min.) was followed by 23 cycles of denaturation at 95° C. (30 sec.), annealing at 53° C. (45 sec.), and extension at 68° C. (2.5 min), with a final extension of 68° C. (5 min.). PCR products were purified from primers and unincorporated nucleotides using 96-well purification columns (Millipore) and the product diluted to give peak heights in the 1000 to 3000 scalar range to ensure accurate assessment of peak area on an ABI 3100 using Genotyper software.

TABLE 1

Mutation Detection Primers for
Parkin Gene Analysis

| | | |
|---|---|---|
| EXON | PRIMER SEQUENCE | PRODUCT SIZE |

| | | |
|---|---|---|
| 1 | F 5′-GCGCGGCTGGCGCCGCTGCGCGCA-3′ | 112 |
| | (SEQ ID NO:15) | |
| | R 5′-GCGGCGCAGAGAGGCTGTAC-3′ | |
| | (SEQ ID NO:16 | |
| 2 | F 5′-ATGTTGCTATCACCATTTAAGGG-3′ | 308 |
| | (SEQ ID NO:17) | |
| | R 5′-AGATTGGCAGCGCAGGCGGCATG-3′ | |
| | (SEQ ID NO:18) | |
| 3 | F 5′-CTTGCTCCCAAACAGAATT-3′ | 314 |
| | (SEQ ID NO:19) | |
| | R 5′-AGGCCATGCTCCATGCAGACTGC-3′ | |
| | (SEQ ID NO:20) | |
| 4 | F 5′-ACAAGCTTTTAAAGAGTTTCTTGT-3′ | 261 |
| | (SEQ ID NO:21) | |
| | R 5′-AGGCAATGTGTTAGTACACA-3′ | |
| | (SEQ ID NO:22) | |
| 5 | F 5′-ACATGTCTTAAGGAGTACATTT-3′ | 227 |
| | (SEQ ID NO:23) | |
| | R 5′-TCTCTAATTTCCTGGCAAACAGTG-3′ | |
| | (SEQ ID NO:24) | |
| 6 | F 5′-CTGTGGAAACATTTAGAGG-3′ | 256 |
| | (SEQ ID NO:25) | |
| | R 5′-GAGTGATGCTATTTTTAGATCCT-3′ | |
| | (SEQ ID NO:26) | |
| 7 | F 5′-TGCCTTTCCACACTGACAGGTACT-3′ | 239 |
| | (SEQ ID NO:27) | |
| | R 5′-TCTGTTCTTCATTAGCATTAGAGA-3′ | |
| | (SEQ ID NO:28) | |
| 8 | F 5′-GTGATTAATTCTTCTTTCCA-3′ | 148 |
| | (SEQ ID NO:29) | |
| | R 5′-ACTGTCTCATTAGCGTCTATCTT-3′ | |
| | (SEQ ID NO:30) | |
| 9 | F 5′-GGGTGAAATTTGCAGTCAGT-3′ | 278 |
| | (SEQ ID NO:31) | |
| | R 5′-AATATAATCCCAGCCCATGTGCA-3′ | |
| | (SEQ ID NO:32) | |
| 10 | F 5′-ATTGCCAAATGCAACCTMTGTC-3′ | 165 |
| | (SEQ ID NO:33) | |
| | R 5′-TTGGAGGAATGAGTAGGGCATT-3′ | |
| | (SEQ ID NO:34) | |
| 11 | F 5′-ACAGGGAACATAAACTCTGATCC-3′ | 303 |
| | (SEQ ID NO:35) | |
| | R 5′-CAACACACCAGGCACCTTCAGA-3′ | |
| | (SEQ ID NO:36) | |
| 12 | F 5′-GTTTGGGAATGCGTGTTTT-3′ | 255 |
| | (SEQ ID NO:37) | |
| | R 5′-AGAATTAGAAAATGAAGGTAGACA-3′ | |
| | (SEQ ID NO:38) | |

12 cases were found to be heterozygous for a single mutation, and nine cases were confirmed to have a single mutation. Mutations detected were as follows: Ex 11 1326 G to T (Glu 409 Stop); Ex 12 1422 T to C (Cys 441Arg); Ex 7 951 G to C (Gly284Arg); Ex 2 202 A to G (Q34R); Int5 +17 A to C; Int5 +2T to A; Int 7 −1G to C; and Ex 3 insertion of CCA after position 500. Additional mutations include a deletion of exon 1; a duplication of exon 2; a duplication of exon 4; a deletion of exons 3-4-5; a deletion of exons 4-5-6-7; and a deletion of exons 7-8-9.

C. Promoter Screening

All 20 patients were sequenced 1 kb through the Parkin gene core promoter (SEQ ID. NO:10), 5′ of the G at position 1 (5′-GGCCTGGAGG, “G+1” underlined; SEQ ID NO:13) up to and including the start of transcription. In addition, 5 kb (SEQ ID. NO:1, Accession No. AF350258) was sequenced upstream of Parkin exon one for the 9 confirmed heterozygous cases. Primers are listed in Table 2, entitled “Primers for Parkin Promoter Analysis.”

TABLE 2
Primers for Parkin Promoter Analysis

| | | |
|---|---|---|
| | | Position in |
| Pair | Primer Sequence | Promoter |

| | | |
|---|---|---|
| 1 | F 5′-CTCGTAGTGCCCAGGTTGATCC-3′ | +348 |
| | (SEQ ID NO:39) | |
| | R 5′-CCACGTACCTATCATGGTCACTGG-3′ | −112 |
| | (SEQ ID NO:40) | |
| 2 | F 5′-GGCCAACCTCTGTAAATCTCGTG-3′ | −695 |
| | (SEQ ID NO:41) | |
| | R 5′-TTCAGGCCCAGCAATCTTACGTC-3′ | +146 |
| | (SEQ ID NO:42) | |
| 3 | F 5′-TTCCCGGTTGTATATCAGCTCATG-3′ | +1036 |
| | (SEQ ID NO:43) | |
| | R 5′AGACCCTGAGCTTAAACAAATGCC-3′ | +487 |
| | (SEQ ID NO:44) | |
| 4 | F 5′-AATAACTCAGATCTTCCCAGGGTG-3′ | +1535 |
| | (SEQ ID NO:45) | |
| | R 5′-ACTCAGCAAAGGGCCTTATAGAAG-3′ | +974 |
| | (SEQ ID NO:46) | |
| 5 | F 5′CATTTGGCAATACAGAAACATCAG-3′ | +2090 |
| | (SEQ ID NO:47) | |
| | R 5′-GCAACTGTCTGGGAATGAGGC-3′ | +1452 |
| | (SEQ ID NO:48) | |
| 6 | F 5′-TATAAACGGTATTGTCCAGCCTTC-3′ | +2452 |
| | (SEQ ID NO:49) | |
| | R 5′-ATCAGCAATACCATAACCATTCAG-3′ | +1882 |
| | (SEQ ID NO:50) | |
| 7 | F 5′-TCTGCTTGCACAGCCCATTTG-3′ | +2846 |
| | (SEQ ID NO:51) | |
| | R 5′-GCTAAGCACAGTTCTGGGATTTGG-3′ | +2330 |
| | (SEQ ID NO:52) | |
| 8 | F 5′-TCAACTTCTCTGTCACCATAACCC-3′ | +3276 |
| | (SEQ ID NO:53) | |
| | R 5′-AACATTCCAATGCTCTTCCACC-3′ | +2694 |
| | (SEQ ID NO:54) | |
| 9 | F 5′-ATCCCAAACATTTCAATCCAAGG-3′ | +3601 |
| | (SEQ ID NO:55) | |
| | R 5′GCCCATGACCAGAAACTAGTAACC-3′ | +3148 |
| | (SEQ ID NO:56) | |
| 10 | F 5′-CTTATCTGAAATGCTTGGGACCAG-3′ | +3936 |
| | (SEQ ID NO:57) | |
| | R 5′-GAACCTGGCGTGACCATCAG-3′ | +3500 |
| | (SEQ ID NO:58) | |
| 11 | F 5′-CTCTGCTTCCACTTTCCTCCTTC-3′ | +4296 |
| | (SEQ ID NO:59) | |
| | R 5′-CGCCATGTTATATCAGGGACTTG-3′ | +3776 |
| | (SEQ ID NO:60) | |
| 12 | F 5′-GTCCCAGCCTCTCTTGCAACTAG-3′ | +4693 |
| | (SEQ ID NO:61) | |
| | R 5′-AGGAGCATGTTTGTTCTTTGCATC-3′ | +4187 |
| | (SEQ ID NO:62) | |
| 13 | F 5′-GGGAGTCAACCAATTGATAGGTG-3′ | +4988 |
| | (SEQ ID NO:63) | |
| | R 5′-AGAATGAGGCAGGAAGAAATGAAG-3′ | +4555 |
| | (SEQ ID NO:64) | |

The frequency of all single nucleotide variants (e.g., SNPs) identified was assessed in patient DNA. Nine single nucleotide promoter variants were identified. See Table 3, entitled “Promoter Polymorphisms in Parkin.” SNP heterozygosity in a control sample of fifty Northern European individuals was also examined.

TABLE 3
Promoter Polymorphisms in Parkin

| | | | | |
|---|---|---|---|---|
| | | Sequence | | |
| | Position in | Adjacent to | | |
| Variant # | Promoter | Variant | Restriction Site | Frequency (het) |

| | | | | |
|---|---|---|---|---|
| 1 | −227 | aaaggtaRgcctccc | StuI | 5% G (0.10) |
| | | (SEQ ID NO:65) | | |
| 2 | −258 | aggacctKggctaga | AlwNI | 14% T (0.24) |
| | | (SEQ ID NO:66) | | |
| 3 | −1511 | cagggtgYaaattac | — | <1% C (0.02) |
| | | (SEQ ID NO:67) | | |
| 4 | −2605 | catacacRtcctgaa | FokI | 41% A (0.48) |
| | | (SEQ ID NO:68) | | |
| 5 | −2983 | catgaaaYttttgtt | Tsp509I | 16% C (0.27) |
| | | (SEQ ID NO:69) | | |
| 6 | −3030 | cctgcaaYgaaataa | BsrDI | 15% T (0.26) |
| | | (SEQ ID NO:70) | | |
| 7 | −3228 | cttatcaYgaagcaa | BspHI | 13% T (0.23) |
| | | (SEQ ID NO:71) | | |
| 8 | −3807 | gcatctgMagatttt | MboII | 46% C (0.50) |
| | | (SEQ ID NO:72) | | |
| 9 | −4578 | aaatgaaRagcaaac | EarI | 14% G (0.24) |
| | | (SEQ ID NO:73) | | |

### Example 2

### Functional Association of Parkin Gene Promoter with Idiopathic PD

The polymorphic variability identified within the Parkin gene promoter was examined to determine if one or more of the SNPs was associated with idiopathic PD.

A. PD Patients and Controls

Cases with PD and controls were derived from an ongoing study of epidemiology and genetics of PD at Mayo Clinic, Rochester, Minn. A total of 319 unrelated PD patients and 196 controls were included. All subjects were examined using a standardized clinical protocol by one of 3 movement disorder specialists and had at least two of four cardinal signs (bradykinesia, rigidity, rest tremor, and postural instability) of PD. The study was approved by the Mayo Institutional Review Board and informed consent was obtained from each subject at the time of blood drawing. Blood samples were processed via the Purgene procedure (Gentra Systems, Minneapolis, Minn.) to extract DNA.

B. Genetic Analysis

Variants were determined using a standard RFLP protocol by first amplifying 25 ng of genomic DNA using the promoter primers set forth in Table 3 above using a 60-50° C. touchdown protocol over 35 cycles. PCR products were then digested with a restriction enzyme (e.g., StuI for the −227 variant and AlwNI for the −258 variant). Enzymes were purchased from New England Biolabs, Beverly Mass. Digested products were analyzed on 3% agarose gels stained with ethidium bromide.

C. Statistical Analysis

The association of the candidate gene with PD was measured by odds ratios (ORs), which closely approximate the relative risk in rare disease. ORs were adjusted for sex (M v. F) using logisitic regression models. ORs were also adjusted for age at examination where appropriate. For each OR, a 95% Confidence Interval (CI) was computed, and a two-sided statistical test was performed at an α-level of 0.05. All analyses were performed using SAS software (Cary, N.C.).

Genotype distributions of the −258 variant, particularly the −258 G allele, demonstrated evidence of association with PD [odds ratio (OR)=1.52; 95% confidence interval (CI)=1.03-2.28, p=0.04.] See Table 4, entitled “−258 T/G Variant Association.” Stratifying PD cases by median age (71 years) showed a significant association with the older-onset group (>71 years). The −258 G allele was observed in 19% of controls and 25% of late-onset PD cases (>71 years).

TABLE 4
−258 T/G Variant Association

| | | | | | OR (95% CI)* |
|---|---|---|---|---|---|
| | | Genotype frequency, No. (%) | | | T/T vs. |
| Sample or stratum | No. | T/T | T/G | G/G | T/G plus G/G |
| Total Controls | 184 | 123 (66.9) | 54 (29.4) | 7 (3.8) | 1.00 (reference) |
| Total Cases | 296 | 171 (57.8) | 112 (37.8) | 13 (4.4) | 1.52 (1.03-2.28) |
| Controls, age at exam ≦ 71‡ | 79 | 52 (65.8) | 25 (31.7) | 2 (2.5) | 1.00 (reference) |
| Cases, age at exam ≦ 71‡ | 162 | 98 (60.5) | 56 (34.6) | 8 (4.9) | 1.28 (0.71-2.34) |
| Controls, age at exam > 71‡ | 105 | 71 (67.6) | 29 (27.6) | 5 (4.8) | 1.00 (reference) |
| Cases, age at exam > 71‡ | 134 | 73 (54.5) | 56 (41.8) | 5 (3.7) | 1.74 (1.02-2.99) |
| Controls, Europeans | 146 | 97 (66.4) | 44 (30.1) | 5 (3.4) | 1.00 (reference) |
| Cases, Europeans | 243 | 136 (56.0) | 94 (38.7) | 13 (5.4) | 1.62 (1.04-2.53) |

*Odds ratios were adjusted for sex and age at examination in logistic regression models. Analyses stratified by sex were adjusted for age at examination only, and analyses stratified by age at examination were adjusted for sex only.
‡Age at onset and age at examination were highly correlated among cases (Pearson's correlation coefficient = 0.88; p = 0.0001); therefore, age at examination was used as a surrogate for age at onset. Age at examination was available for both cases and controls.

D. DNA-Binding Analysis

To assess the functional potential of genetic variability in the Parkin core promoter (SEQ ID NO:10), in silico sequence analysis was used to predict the presence of DNA-binding domains about the −258 and −227 variant regions. See Quandt et al., “MatInd and Matinspector: new fast and versatile tools for detection of consensus matches in nucleotide sequence data,” Nucleic Acids Res. 23:4878-4884 (1995). Using MatInspector v2.2 (http://transfac.gb.de/), an NF1-like protein binding site was predicted near the −258 variant. A ‘T’ at position −258 generated an NF1-like site with a MatInspector core similarity of 1.00 and an affinity of 0.935, whereas a ‘G’ at position −258 generated a core similarity of 0.748 and an affinity score of 0.745. The in silico results suggested that the −258 T allele was more likely to bind NF1-like proteins than the −258 G allele.

The NF1-like sequence consensus motif (TTGGC) in the Parkin core promoter (SEQ ID NO:10) had been previously described to regulate the transcription of the regucalcin gene. To examine if the TTGGC motif could bind protein derived from human substantia nigra, including proteins important in the regulation of the Parkin gene, electromobility shift assays were used to determine protein-binding affinity. Nuclear protein was derived from human fresh-frozen substantia nigra tissue using the Sigma Nu-CLEAR kit (Sigma Life Sciences), according to the manufacturer's suggested protocol. Probes to detect the −258 variant were made by Invitrogen (Carlsbad, Calif.) and cartridge purified to select for full-length oligonucleotides. Specific primers used were as follows:

| | |
|---|---|
| Forward −258 T variant = 5′-GGCAGGACCTTGGCTAGAGCTG-3′; | (SEQ ID NO:74) |
| Reverse −258 T variant = 5′-CAGCTCTAGCCAAGGTCCTGCC-3′; | (SEQ ID NO:75) |
| Forward −258 G variant = 5′-GGCAGGACCTGGGCTAGAGCTG-3′; | (SEQ ID NO:76) |
| and | |
| Reverse −258 G variant = 5′-CAGCTCTAGCCCAGGTCCTGCC-3′. | (SEQ ID NO:77) |

The two −258 variant-specific double-stranded oligonucleotides were generated by heating the complementary oligonucleotides in a high-salt solution (10 mM Tris-HCl, pH 7.5, 1 mM EDTA, and 100 mM NaCl) at 65° C. for 15 in., and then allowing the solutions to cool to room temperature. Double-stranded DNAs were labeled using [γ-³²P]dATP (3000mCi/mmol, NEN) and T4 polynucleotide kinase (Promega, Madison, Wis.), and radioactivity was counted by liquid scintillation. The Gel-Shift Assay System® (Promega) was employed using the manufacturer's protocol, and allele-specific competition reactions were carried out in tandem. Products were electrophoresed in Novex 6% DNA retardation gels in 0.5×TBE running buffer at 100V, and gels were dried and visualized using Kodak Biomax® film with one intensifier screen at −70° C. overnight.

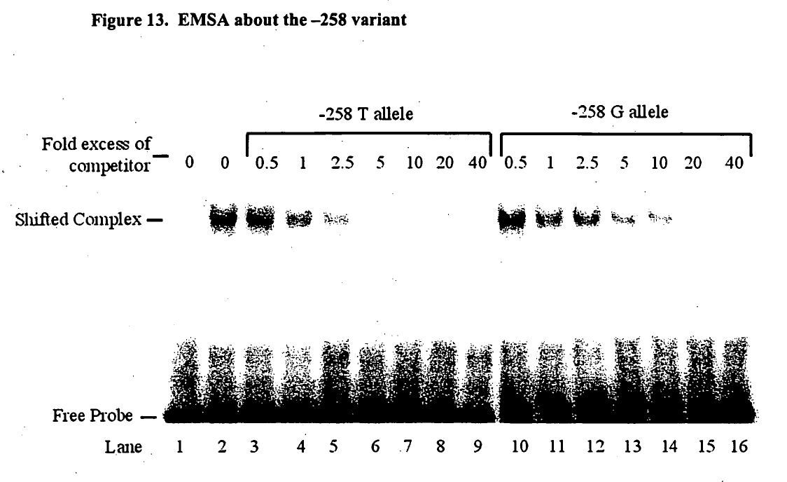

Gel-shift experiments verified that the sequence about position −258 bound nuclear protein derived from human substantia nigra. Labeled probes (both the −258 T allele and the -258 G allele) were shifted when incubated with nuclear protein derived from human substantia nigra. See FIG. 13. Similar results were obtained with nuclear protein derived from M17 and HEK nuclear protein extracts.

To determine the effect of the −258 T/G allele on protein binding, a competition assay was used to measure the effectiveness of the two alleles as competitors for protein binding. Specificity of the protein-probe interaction was examined by measuring the reduction of the shifted complex upon addition of unlabeled probe. Both the T and G allele-specific unlabeled probes completely competed away the shifted complex at 40-molar excess to labeled T allele probe. However, at lower concentrations of competitor probe, the G allele did not compete the shifted complex as efficiently as the T allele, suggesting that the T to G alteration may reduce nuclear protein-binding affinity. See FIG. 13.

E. Effect of Mutations on Transcription Regulation

A dual-luciferase assay was used to assess the in vivo effects of the −258 T/G allele on transcription regulation. Three parkin core promoter constructs, containing the −258 T allele, the −258 G allele, or an NF1-A1 consensus site knockout, were amplified from BAC DNA containing parkin exon 1, using primers with internal restriction sites for cloning. The knockout promoter fragment was designed with multiple mutations across the consensus TTGGC NF1-A1-binding motif; this promoter fragment had been previously shown to negate interactions with nuclear protein (Misawa et al., “Involvement of hepatic nuclear factor I binding motif in transcriptional regulation of Ca2+-binding protein regucalcin gene,” Biochem. Biophys. Res. Commun. 269:270−278 (2000)). Primers used were as follows:

| | | |
|---|---|---|
| Forward −258 T: | 5′-GGAAGAGGTACCGACCTTGGCTA-3′; | (SEQ ID NO:78) |
| Forward −258 G: | 5′-GGAAGAGGTACCGACCTGGGCTA-3′; | (SEQ ID NO:79) |
| Forward - knockout: | 5′-GGGAAGAGGTACCGACCTGTTGTA-3′; | (SEQ ID NO:80) |
| and | | |
| Reverse (all): | 5′-CGTGTTGACCAGTCGCTAGCCA-3′. | (SEQ ID NO:81) |

PCR was performed using a 65-55° C. touchdown protocol, with Taq DNA polymerase (Qiagen) and 1 ng of BAC DNA. PCR products and the luciferase-containing pGL3-Basic vector (Promega) were digested with KpnI and NheI (Roche Biochemicals) and purified (Qiagen) according to the manufacturer's conditions. Vector arms were dephosphorylated (CIP, Promega) and ligated to digested PCR fragments (DNA Rapid Ligation Kit®, Roche Biochemicals). Constructs were subcloned into DH5α cells (Life Technologies). Single colonies were miniprepped (Qiagen) and the insert was verified by sequence analysis.

Human dopaminergic neuroblastoma cells (BE(2)-M17) and human embryonic kidney cells (HEK-293T) were cultured in Opti-MEM (Life Technologies) supplemented with 10% FBS, penicillin (100 units/ml), and streptomycin (100 μg/ml). Cells were plated 24 h prior to transfection into 24-well culture plates at 80% confluence and maintained in an atmosphere of 5% CO₂ at 37° C. Transfection was performed with Fugene (Roche Biochemicals), using 0.2 μg of DNA per well, in a 1:3 ratio of DNA:Fugene reagent, and added to cells in serum-free media for 12 h.

Luciferase-containing constructs (pGL3) were co-transfected with phRL-TK synthetic renilla vector (Promega) to control for transfection efficiency, in a molar ratio of 1:100 (phRL-TK versus pGL3). Forty hours after transfection, cells were gently rinsed with PBS and then harvested with Passive Lysis buffer (Promega). The Dual Luciferase Systems (Promega) was used to assay promoter activity according to the manufacturer's protocol, and experiments were repeated in six independent wells. SV40 was used as a control for promoter activity. Readings were taken in duplicate on a Turner Designs 20/20 Single Injector Luminometer.

The −258 G allele reduced luciferase activity by approximately 25% relative to the −258 T allele. The NF1-A1 knockout vector also reduced luciferase activity by 25%, illustrating the importance of the −258 nucleotide in transcription regulation.

A number of embodiments of the invention have been described. Nevertheless, it will be understood that various modifications may be made without departing from the spirit and scope of the invention. Accordingly, other embodiments are within the scope of the following claims.

1. An isolated nucleic acid molecule comprising a Parkin nucleic acid sequence, wherein said nucleic acid molecule is at least ten nucleotides in length, and wherein said Parkin nucleic acid sequence comprises a nucleotide sequence variant at a position selected from the group consisting of:
a) position −227, −258, −1511, −2605, −2983, −3030, −3228, −3807, or −4578 relative to the guanine (position +1) of the transcription start site of the Parkin promoter given in SEQ ID NO: 1;
b) position 1326 relative to the Tat position +1 of SEQ ID NO:11;
c) position 1422 relative to the T at position +1 of SEQ ID NO:11;
d) position +2 or position +17 relative to the guanine (position +1) in the splice donor site of Intron 5 in SEQ ID NO: 4;
e) position +1 in the splice donor site of Intron 7 within SEQ ID NO:5;
f) position 951 relative to the T at position +1 of SEQ ID NO:11;
g) position 202 relative to the T at position +1 of SEQ ID NO:1; and
h) position 500 relative to the T at position +1 of SEQ ID NO:11.

2. The isolated nucleic acid of claim 1, wherein said nucleotide sequence variant is a nucleotide substitution.

3. The isolated nucleic acid of claim 1, wherein said nucleotide sequence variant is a nucleotide insertion.

4. The isolated nucleic acid of claim 1, wherein said nucleotide sequence variant is a nucleotide deletion.

5. The isolated nucleic acid of claim 1, wherein said nucleotide sequence variant is a guanine substitution for adenine at position −227 relative to the guanine of the transcription start site of the Parkin promoter given in SEQ ID NO: 1.

6. The isolated nucleic acid of claim 1, wherein said nucleotide sequence variant is a guanine substitution for thymine at position −258 relative to the guanine of the transcription start site of the Parkin promoter given in SEQ ID NO: 1.

7. The isolated nucleic acid of claim 1, wherein said nucleotide sequence variant is a cytosine substitution for thymine at position −1511 relative to the guanine of the transcription start site of the Parkin promoter given in SEQ ID NO: 1.

8. The isolated nucleic acid of claim 1, wherein said nucleotide sequence variant is a guanine substitution for adenine at position −2605 relative to the guanine of the transcription start site of the Parkin promoter given in SEQ ID NO: 1.

9. The isolated nucleic acid of claim 1, wherein said nucleotide sequence variant is a cytosine substitution for thymine at position −2983 relative to the guanine of the transcription start site of the Parkin promoter given in SEQ ID NO: 1.

10. The isolated nucleic acid of claim 1, wherein said nucleotide sequence variant is a cytosine substitution for thymine at position −3030 relative to the guanine of the transcription start site of the Parkin promoter given in SEQ ID NO: 1.

11. The isolated nucleic acid of claim 1, wherein said nucleotide sequence variant is a thymine substitution for cytosine at position −3228 relative to the guanine of the transcription start site of the Parkin promoter given in SEQ ID NO: 1.

12. The isolated nucleic acid of claim 1, wherein said nucleotide sequence variant is a adenine substitution for cytosine at position −3807 relative to the guanine of the transcription start site of the Parkin promoter given in SEQ ID NO: 1.

13. The isolated nucleic acid of claim 1, wherein said nucleotide sequence variant is a adenine substitution for guanine at position −4578 relative to the guanine of the transcription start site of the Parkin promoter given in SEQ ID NO: 1.

14. The isolated nucleic acid of claim 1, wherein said nucleotide sequence variant is a thymine substitution for guanine at position 1326 relative to the T at position +1 in SEQ ID NO:11.

15. The isolated nucleic acid of claim 1, wherein said nucleotide sequence variant is a cytosine substitution for thymine at position 1422 relative to the T at position +1 in SEQ ID NO:11.

16. The isolated nucleic acid of claim 1, wherein said nucleotide sequence variant is an adenine substitution for thymine at the +2 position relative to the guanine in the splice donor site of Intron 5 within SEQ ID NO: 4.

17. The isolated nucleic acid of claim 1, wherein said nucleotide position variant is a cytosine substitution for guanine at position +1 of the splice donor site of Intron 7 within SEQ ID NO: 5.

18. The isolated nucleic acid of claim 1, wherein said nucleotide position variant is a cytosine substitution for guanine at position 951 relative to the T at position +1 of SEQ ID NO. 11.

19. The isolated nucleic acid of claim 1, wherein said nucleotide position variant is a guanine substitution for adenine at position 202 relative to the T at position +1 SEQ ID NO. 11.

20. The isolated nucleic acid of claim 1, wherein said nucleotide position variant is a cytosine substitution for adenine at position +17 relative to the guanine in the splice donor site of Intron 5 within SEQ ID NO: 4.

21. The isolated nucleic acid of claim 1, wherein said nucleotide position variant is a nucleotide insertion of the nucleotides 5′-CCA-3′ after position 500 relative to the T at position +1 of SEQ ID NO:11.

22. The isolated nucleic acid of claim 1, wherein said Parkin nucleic acid sequence comprises a sequence variant associated with Parkinson's disease.

23. The isolated nucleic acid of claim 22, wherein said Parkinson's disease is autosomal recessive juvenile parkinsonism.

24. The isolated nucleic acid of claim 22, wherein said Parkinson's disease is early-onset Parkinson's disease.

25. The isolated nucleic acid of claim 22, wherein said Parkinson's disease is juvenile-onset Parkinson's disease.

26. The isolated nucleic acid of claim 22, wherein said Parkinson's disease is late onset Parkinson's disease.

27. The isolated nucleic acid of claim 26, wherein said sequence variant associated with late-onset Parkinson's disease is a guanine substitution for thymine at position −258 relative to the guanine of the transcription start site of the Parkin promoter given in SEQ ID NO: 1.

28. An isolated nucleic acid encoding a Parkin polypeptide, wherein said polypeptide comprises a Parkin amino acid sequence variant relative to the amino acid sequence of SEQ ID NO: 9, and wherein said amino acid sequence variant is at residue 34, 284, or 441.

29. The isolated nucleic acid of claim 28, wherein said amino acid sequence variant is an Arg at residue 441.

30. The isolated nucleic acid of claim 28, wherein said amino acid sequence variant is an Arg at residue 34.

31. The isolated nucleic acid of claim 28, wherein said amino acid sequence variant is an Arg at residue 284.

32. An isolated nucleic acid encoding a Parkin polypeptide, wherein said polypeptide consists of residues 1-408 relative to the amino acid sequence of SEQ ID NO: 9.

33. An isolated nucleic acid encoding a Parkin polypeptide, wherein said polypeptide comprises a Parkin amino acid sequence variant relative to the amino acid sequence of SEQ ID NO:9, and wherein said amino acid sequence variant is an insertion of an amino acid after amino acid residue 133 of SEQ ID NO:9.

34. The isolated nucleic acid of claim 33, wherein said amino acid sequence variant is an insertion of a Pro after amino acid residue 133.

35. An isolated Parkin polypeptide, said polypeptide having an amino acid sequence variant relative to the amino acid sequence of SEQ ID NO:9, and wherein said amino acid sequence variant is selected from the group consisting of:
a) an Arg at residue 34;
b) an Arg at residue 284;
c) an Arg at residue 441; and
d) an insertion of a proline after amino acid position 133 of SEQ ID NO:9.

36. The isolated polypeptide of claim 35, wherein an activity of said polypeptide is altered relative to wild type Parkin polypeptide of SEQ ID NO:9.

37. A method for determining the susceptibility of a subject to Parkinson's disease, said method comprising providing a nucleic acid sample from said subject and determining if a Parkin nucleotide sequence variant at position −258 relative to the guanine (position +1) of the transcription start site of the Parkin promoter (SEQ ID NO: 1) is present or absent in said nucleic acid sample, wherein the presence of said nucleotide sequence variant is associated with increased susceptibility of said subject to Parkinson's disease.

38. The method of claim 37, wherein said subject is a mammal.

39. The method of claim 37, wherein said subject is a human.

40. The method of claim 37, wherein said nucleic acid sample is genomic DNA.

41. The method of claim 37, wherein said nucleic acid sample is cDNA.

42. The method of claim 37, wherein said determining step is performed by
a) contacting said nucleic acid sample with an article of manufacture comprising a substrate, said substrate comprising a plurality of discrete regions, wherein each of said regions comprises a different population of nucleic acid molecules, wherein said nucleic acid molecules are at least 10 nucleotides in length, wherein at least one said population of nucleic acid molecules comprises a guanine substitution for thymine at position −258 relative to the guanine (position +1) of the transcription start site of the Parkin promoter given in SEQ ID NO: 1; and
b) determining if said nucleic acid sample is bound to said article of manufacture.

43. The method of claim 42, wherein at least one of said population comprises a wild-type Parkin nucleic acid sequence.

44. The method of claim 37, further comprising detecting the presence or absence of one or more additional Parkin nucleotide sequence variants.

45. The method of claim 44, wherein said one or more additional Parkin nucleotide sequence variants is at a position selected from the group consisting of:
a) position −227, −1511, −2605, −2983, −3030, −3228, −3807, or −4578 relative to the guanine (position +1) of the transcription start site of the Parkin promoter given in SEQ ID NO: 1;
b) position 1326 relative to the T at position +1 of SEQ ID NO:11;
c) position 1422 relative the T at position +1 of SEQ ID NO:11;
d) position +2 or position +17 relative to the guanine (position +1) in the splice donor site of Intron 5 within SEQ ID NO:4;
e) position +1 in the splice donor site of Intron 7 within SEQ ID NO:5;
f) position 951 relative to the T at position +1 of SEQ ID NO:11;
g) position 202 relative to the T at position +1 of SEQ ID NO:11; and
h) position 500 relative to the T at position +1 of SEQ ID NO:11.

46. The method of claim 45, wherein said one or more additional Parkin nucleotide sequence variants is a nucleotide substitution of a wild type Parkin nucleic acid sequence or a nucleotide insertion at a wild type Parkin nucleic acid sequence selected from the group consisting of:
a) a guanine substitution for adenine at position −227 relative to the guanine of the transcription start site of the Parkin promoter in SEQ ID NO:1;
b) a cytosine substitution for thymine at position −1511 relative to the guanine of the transcription start site of the Parkin promoter in SEQ ID NO:1;
c) a guanine substitution for adenine at position −2605 relative to the guanine of the transcription start site of the Parkin promoter in SEQ ID NO:1;
d) a cytosine substitution for thymine at position −2983 relative to the guanine of the transcription start site of the Parkin promoter in SEQ ID NO:1;
e) a cytosine substitution for thymine at position −3030 relative to the guanine of the transcription start site of the Parkin promoter in SEQ ID NO:1;
f) a thymine substitution for cytosine at position −3228 relative to the guanine of the transcription start site of the Parkin promoter in SEQ ID NO:1;
g) an adenine substitution for cytosine at position −3807 relative to the guanine of the transcription start site of the Parkin promoter in SEQ ID NO:1;
h) an adenine substitution for guanine at position −4578 relative to the guanine of the transcription start site of the Parkin promoter in SEQ ID NO:1;
i) a thymine substitution for guanine at position 1326 relative to the T at position +1 of SEQ ID NO:11;
j) a cytosine substitution for thymine at position 1422 relative to the T at position +1 of SEQ ID NO:11;
k) an adenine substitution for thymine at the +2 position relative to the guanine in the splice donor site of Intron 5 in SEQ ID NO:4;
l) a cytosine substitution for adenine at position +17 relative to the guanine in the splice donor site of Intron 5 in SEQ ID NO:4;
m) a cytosine substitution for guanine at position 951 relative to the T at position +1 of SEQ ID NO:11;
n) a guanine substitution for adenine at position 202 relative to T at position +1 of SEQ ID NO:11;
o) a cytosine substitution for guanine at position +1 in the splice donor site of Intron 7 in SEQ ID NO:5; and
p) an insertion of the nucleotides 5′-CCA-3′ after position 500 relative to the T at position +1 of SEQ ID NO:11.

47. A method for diagnosing Parkinson's disease in a subject, said method comprising providing a nucleic acid sample from said subject, and determining whether said nucleic acid sample comprises a Parkin nucleotide sequence variant at position −258 relative to the guanine (position +1) of the transcription start site of the Parkin promoter given in SEQ ID NO: 1, wherein the presence of said Parkin nucleotide sequence variant is diagnostic of Parkinson's disease.

48. The method according to claim 47, wherein said Parkin nucleotide sequence variant at position −258 relative to the guanine of the transcription start site of the Parkin promoter is a guanine substitution for thymine at position −258.

49. An article of manufacture comprising a substrate, wherein said substrate comprises a population of isolated nucleic acid molecules, wherein each of said nucleic acid molecules is 10 to 1000 nucleotides in length, wherein said population contains a plurality of Parkin nucleic acid sequence variants, and wherein at least one of said Parkin nucleic acid sequence variants is independently selected from the group consisting of:
a) position −227, −258, −1511, −2605, −2983, −3030, −3228, −3807, or −4578 relative to the guanine (position +1) of the transcription start site of the Parkin promoter given in SEQ ID NO: 1;
b) position 1326 relative to the T at position +1 of SEQ ID NO:11;
c) position 1422 relative to the T at position +1 of SEQ ID NO:11;
d) position +2 or position +17 relative to the guanine (position +1) in the splice donor site of Intron 5 within SEQ ID NO:4;
e) position +1 in the splice donor site of Intron 7 within SEQ ID NO: 5;
f) position 951 relative to the T at position +1 of SEQ ID NO:11;
g) position 202 relative to the T at position +1 of SEQ ID NO:11; and
h) position 500 relative to the T at position +1 of SEQ ID NO:11.

50. The article of manufacture according to claim 49, wherein at least one of said Parkin nucleic acid sequence variants is a guanine substitution for thymine at position −258 relative to the guanine of the transcription start site of the Parkin promoter given in SEQ ID NO: 1.

51. An article of manufacture comprising a substrate, said substrate comprising a plurality of discrete regions, wherein each of said regions comprises a different population of nucleic acid molecules, wherein at least one of said population of nucleic acid molecules comprises a Parkin nucleotide sequence variant, and wherein said Parkin nucleotide sequence variant comprises a guanine substitution for thymine at position −258 relative to the guanine (position +1) of the transcription start site of the Parkin promoter given in SEQ ID NO: 1.

52. An isolated nucleic acid molecule comprising a Parkin nucleic acid sequence, wherein said nucleic acid molecule is at least ten nucleotides in length, and wherein said Parkin nucleic acid sequence comprises a nucleotide sequence variant at a position within the Parkin core promoter set forth in SEQ ID NO: 10.

53. The isolated nucleic acid of claim 52, wherein said nucleotide sequence variant is at a position selected from the group consisting of position −259, −258, −257, −256, −255, −254, or −253 relative to the guanine (position +1) of the transcription start site of the Parkin core promoter given in SEQ ID NO: 10.

54. The isolated nucleic acid of claim 52, wherein said nucleotide sequence variant affects the binding of an NF1-like protein to said isolated nucleic acid.

55. The isolated nucleic acid of claim 54, wherein the binding of said NF1-like protein is reduced relative to binding of said NF1-like protein to a corresponding wild-type Parkin core promoter sequence.

56. The isolated nucleic acid of claim 52, wherein said nucleotide sequence variant affects the binding of a protein present in human substantia nigra to said isolated nucleic acid.

57. The isolated nucleic acid of claim 56, wherein said binding of said protein in human substantia nigra is reduced relative to binding of said protein to a corresponding wild-type Parkin core-promoter sequence.
